(19) United States
(12) Patent Application Publication
(10) Pub. No.: US 2024/0159753 A1
(43) Pub. Date: May 16, 2024

(54) METHODS FOR THE DETECTION AND TREATMENT OF LUNG CANCER

(71) Applicant: Board of Regents, The University of Texas System, Austin, TX (US)

(72) Inventors: Samir HANASH, Houston, TX (US); Edwin OSTRIN, Houston, TX (US); Ziding FENG, Houston, TX (US)

(21) Appl. No.: 18/193,917

(22) Filed: Mar. 31, 2023

Related U.S. Application Data
(63) Continuation of application No. PCT/US2021/052611, filed on Sep. 29, 2021.
(60) Provisional application No. 63/106,187, filed on Oct. 27, 2020.
(60) Provisional application No. 63/086,865, filed on Oct. 2, 2020.

(51) Int. Cl. G01N 33/574; G01N 33/68

(52) U.S. Cl. CPC G01N 33/57423; G01N 33/57484; G01N 33/6848; G01N 2800/50

(57) ABSTRACT

Provided are methods and related kits for detection of early stage lung cancer, and determination of risk of harboring lung cancer.

### CROSS REFERENCE TO RELATED APPLICATIONS

This application is a bypass continuation of International Application No. PCT/US2021/052611, filed Sep. 29, 2021, which claims the benefit of U.S. Provisional Patent Application No. 63/086,865, filed Oct. 2, 2020, and U.S. Provisional Patent Application No. 63/106,187, filed Oct. 27, 2020, the disclosures of which are incorporated by reference herein in their entireties.

### BACKGROUND

Lung nodules are a common finding on chest computed tomography (CT) scans. A relatively high percentage of subjects in the general population (˜1.9%) have a chest CT done per year, with incidental nodules discovered in some 24-31% similar to the incidence of nodules reported in the National Lung Screening Trial (24%). The risk that a nodule is a cancer largely revolves around its size, with nodules greater than 20 mm often referred for work-up. Recommendations for smaller nodules are to follow them with additional imaging, for instance PET/CT, or short follow up repeated CT scans. Such an approach carries a risk of missing an early-stage lung cancer.

Each year more than 150,000 patients present to their doctors with pulmonary indeterminate nodules. These nodules are defined as lung lesions, less than 3 cm, with clearly defined borders. The majority of these are non-cancerous or benign. The diagnosis of a benign lesion is easier to make when the nodules contain calcifications. Very small nodules (less than 1 cm) are even more likely benign; however, this is challenging to prove with current biopsy and imaging techniques.

Although the majority of indeterminate nodules are benign, some are malignant leading to additional interventions. For patients considered low risk for malignant nodules, current medical practice dictates scans for at least two years to monitor for lung cancer. The time period between identification of a indeterminate nodules and diagnosis is a time of medical surveillance or “watchful waiting” and may induce stress on the patient and lead to significant risk and expense due to repeated imaging studies. If a biopsy is performed on a patient who is found to have a benign nodule, the costs and potential for harm to the patient increase unnecessarily. Major surgery is indicated in order to excise a specimen for tissue biopsy and diagnosis. All of these procedures are associated with risk to the patient including: illness, injury and death as well as high economic costs.

A key unmet clinical need for the management of pulmonary nodules is a non-invasive diagnostic test that discriminates between malignant and benign processes in patients with indeterminate pulmonary nodules (IPNs), especially between 8 mm and 20 mm in size.

### SUMMARY

Provided is a four-protein biomarker panel (protein pro-surfactant protein B (proSFTPB), cancer antigen 125 (CA125), carcinoembryonic antigen (CEA), and cytokeratin-21 fragment (CYFRA 21-1)) for stratifying pulmonary nodules and methods for the use of the panel to stratify the risk of pulmonary nodules discovered by CT scans, either discovered by lung cancer screening or outside of screening. A positive 4MP test can identify pulmonary nodules with a high risk of harboring lung cancer that would otherwise be deemed low risk by current nodule size-based risk calculators. Similarly, a negative 4MP test can identify otherwise-risk nodules that may not require diagnostic work up and be safely followed by radiographic means. The methods use multiple assays of biomarkers contained within a biological sample obtained from a subject.

Also provided is a 2-microRNA (miRNA) panel (miR-320 and miR-210) for improved detection of lung cancer and methods for their use in combination with the 4MP test. The combination of the 2-miRNA panel with the 4MP test can better identify pulmonary nodules with a high risk of harboring lung cancer. The methods use multiple assays of biomarkers contained within a biological sample obtained from a subject.

Also provided is a 3-microRNA (miRNA) panel (miR-320, miR-210, and miR-21) for improved detection of lung cancer and methods for their use in combination with the 4MP test. The combination of the 3-miRNA panel with the 4MP test can better identify pulmonary nodules with a high risk of harboring lung cancer. The methods use multiple assays of biomarkers contained within a biological sample obtained from a subject.

Also provided is a 7-metabolite marker panel (diacetylspermine, diacetyspermidine, acetylspermidine, 1-methyladenosine, n-acetyllactosamine, arginine, and dimethyl-arginine) for improved detection of lung cancer and methods for their use in combination with the 4MP test. The combination of the 7-metabolite panel with the 4MP test can better identify pulmonary nodules with a high risk of harboring lung cancer. The methods use multiple assays of biomarkers contained within a biological sample obtained from a subject.

Also provided are methods of combining both the 2-miRNA panel and the 7-metabolite panel with the 4MP test for improved detection of lung cancer. Also provided are methods of combining both the 3-miRNA panel and the 7-metabolite panel with the 4MP test for improved detection of lung cancer. The combination of the 2-miRNA panel or the 3-miRNA panel and the 7-metabolite panel together with the 4MP test can better identify pulmonary nodules with a high risk of harboring lung cancer. The methods use multiple assays of biomarkers contained within a biological sample obtained from a subject.

### BRIEF DESCRIPTION OF THE DRAWINGS

FIG. 1A to FIG. 1E depict the performance of the 4MP in the Pittsburgh nodule cohort. FIG. 1A: The 4MP shows an AUC of 0.76 (95% CI 0.69-0.82). Three of the markers performed moderately well, including pro-SFTPB (FIG. 1B) with an area under the curve (AUC) of the receiver operative characteristic (ROC) of 0.69 (95% CI 0.62-0.77), CEA (FIG. 1C) at 0.70 (95% CI 0.63-0.77), and CYFRA21-1 (FIG. 1D) at 0.72 (95% CI 0.65-0.80). CA125 (FIG. 1E) did not show statistical significance with an AUC of 0.57 (95% CI 0.49-0.65).

FIG. 2A to FIG. 2E depict the performance of the 4MP in the Pittsburgh cohort by nodule size. FIG. 2A shows a Cox model accounting for age, gender, smoking history, and nodule size showed significant interaction between the 4MP and nodule size but none of the other variables. Compared to nodule size alone, the 4MP moderately improved performance, increasing AUC from 0.86 to 0.90 (p-value of the comparison 0.033). Pro-SFTPB (FIG. 2B) and CYFRA21-1 (FIG. 2D) drove this interaction, showing significantly higher performance in larger nodules. CEA and CA125 (FIG. 2C and FIG. 2E) did not show significant interaction with nodule size.

FIG. 3 depicts the performance of the 4MP combined with a nodule size-based risk model. The 4MP improves performance of a risk model for lung cancer based on nodule size, increasing AUC from 0.86 to 0.89. The black box indicates that this performance improvement is pronounced on the left side of the ROC, indicating an increase in sensitivity at a high specificity.

FIG. 4A and FIG. 4B depict the performance of the composite 4MP in the Southwestern nodule cohort. FIG. 4A: The 4MP shows an AUC of 0.87 (95% CI 0.79-0.96), consistent with its performance in the Pittsburgh nodule cohort. Individual marker performance ranged from CYFRA21-1 at an AUC of 0.63 (95% CI 0.49-0.78), CEA at 0.72 (95% CI 0.59-0.86), to pro-SFTPB at 0.76 (95% CI 0.63-0.88) and CEA at 0.80 (95% CI 0.69-0.91). FIG. 4B: In a subset of nodules <6 mm, the 4MP markedly improved performance of the nodule-size risk model. While nodule size alone predicted cancer with an AUC of 0.57 (95% CI 0.35-0.79), addition of the 4MP increased this to 0.95 (95% CI 0.85-1.000).

FIG. 5A and FIG. 5B are 3D visualizations of the 4MP with nodule size and smoking pack-years. FIG. 5A shows a 3-dimensional projection of the 4MP logistic regression model using nodule size and probability of cancer. The same projection is displayed from above and 3 directions. FIG. 5B shows a similar 3-dimensional projection of the model utilizing pack-years and nodule size and probability of cancer. A lack of slope on the pack-years axis indicates that it lends only small contribution towards cancer prediction.

FIG. 6A and FIG. 6B depict the performance of the 4MP in the Pittsburgh cohort by smoking status. The 4MP and its individual components did not show significant differences between former (FIG. 6A) and current (FIG. 6B) smokers in the Pittsburgh cohort.

FIG. 7A to FIG. 7E depict the 4MP in the Pittsburgh cohort by smoking pack-years. In the Pittsburgh cohort, the 4MP (FIG. 7A) or its individual components (FIG. 7B-FIG. 7E) did not show significant changes based on pack-years of smoking. Curves are plotted based on the Cox model.

FIG. 8 depicts the 4MP in the UTSW cohort combined with a nodule size-based lung cancer risk model. In the UTSW cohort, the 4MP combined with nodule size improved performance over nodule size alone, showing an AUC of 0.86 (95% CI 0.76-0.96) compared to nodule size alone which had an ROC of 0.54 (95% CI 0.37-0.70). Again noted was an improvement in sensitivity at a high specificity (black outline).

FIG. 9 depicts the improved detection of lung cancer for miRNAs miR-320 and miR-210 in combination with the 4MP compared to the 4MP alone, showing an 11% improvement in sensitivity at 95% specificity.

FIG. 10 depicts the improved ability to distinguish lung cancer cases from non-cancer cases in the PLCO cohort (within 1 year of blood draw) when using a panel of 7 cancer-associated metabolites (diacetylspermine, diacetyspermidine, acetylspermidine, 1-methyladenosine, n-acetyllactosamine, arginine, and dimethyl-arginine) compared to the 4MP alone. The combination of the 7-metabolite panel with the 4MP yields an additional improvement in the AUC by 7% compared to the 4MP alone.

FIG. 11 depicts the performance of the 3-miRNA panel. Left panel shows a ROC plot of the performance of the 3-miRNA panel alone for diagnosing lung cancer. Right panel shows a ROC plot of the performance of 3-miRNA panel when combined with the 4MP for diagnosing lung cancer. The blue bar highlights increased sensitivity at high specificity.

### DETAILED DESCRIPTION

Provided is a method of determining the risk of a subject with indeterminate pulmonary nodules for harboring lung cancer, comprising:
obtaining a biological sample from the subject;
measuring the level of CEA in the biological sample;
measuring the level of CA125 in the biological sample;
measuring the level of CYFRA21-1 in the biological sample; and
measuring the level of Pro-SFTPB in the biological sample;
wherein the amount of CEA, CA125, CYFRA21-1, and Pro-SFTPB classifies the subject as being at risk of harboring lung cancer or not at risk of harboring lung cancer.

Also provided is a method of distinguishing benign from malignant pulmonary nodules in a subject, comprising:
obtaining a biological sample from the subject;
measuring the level of CEA in the biological sample;
measuring the level of CA125 in the biological sample;
measuring the level of CYFRA21-1 in the biological sample; and
measuring the level of Pro-SFTPB in the biological sample;
wherein the amount of CEA, CA125, CYFRA21-1, and Pro-SFTPB distinguishes benign pulmonary nodules from malignant pulmonary nodules.

Also provided is a method of determining the risk of a subject with indeterminate pulmonary nodules for harboring lung cancer, comprising:
obtaining a biological sample from the subject;
providing a surface that binds CEA, CA125, CYFRA21-1, and Pro-SFTPB;
incubating the surface with the biological sample;
contacting the surface with a first reporter molecule that binds CEA;
contacting the surface with a second reporter molecule that binds CA125;
contacting the surface with a third reporter molecule that binds CYFRA21-1;
contacting the surface with a fourth reporter molecule that binds Pro-SFTPB;
measuring the amount of the first reporter molecule that is associated with the surface;
measuring the amount of the second reporter molecule that is associated with the surface;
measuring the amount of the third reporter molecule that is associated with the surface;
measuring the amount of the fourth reporter molecule that is associated with the surface;
wherein the amount of the first reporter molecule, the second reporter molecule, the third reporter molecule, and the fourth reporter molecule classifies the subject with indeterminate pulmonary nodules as being at risk of harboring lung cancer or not at risk of harboring lung cancer.

Also provided is a method of distinguishing benign from malignant pulmonary nodules in a subject, comprising:
obtaining a biological sample from the subject;
providing a surface that binds CEA, CA125, CYFRA21-1, and Pro-SFTPB;
incubating the surface with the biological sample;
contacting the surface with a first reporter molecule that binds CEA;
contacting the surface with a second reporter molecule that binds CA125;
contacting the surface with a third reporter molecule that binds CYFRA21-1;
contacting the surface with a fourth reporter molecule that binds Pro-SFTPB;
measuring the amount of the first reporter molecule that is associated with the surface;
measuring the amount of the second reporter molecule that is associated with the surface;
measuring the amount of the third reporter molecule that is associated with the surface;
measuring the amount of the fourth reporter molecule that is associated with the surface;
wherein the amount of the first reporter molecule, the second reporter molecule, the third reporter molecule, and the fourth reporter molecule distinguishes benign pulmonary nodules from malignant pulmonary nodules.

Also provided is a method of determining the risk of a subject with indeterminate pulmonary nodules for harboring lung cancer, comprising:
obtaining a biological sample from the subject;
providing a first surface with means for binding CEA;
providing a second surface with means for binding CA125;
providing a third surface with means for binding CYFRA21-1;
providing a fourth surface with means for binding Pro-SFTPB;
incubating the first surface with the biological sample;
incubating the second surface with the biological sample;
incubating the third surface with the biological sample;
incubating the fourth surface with the biological sample;
contacting the first surface with a first reporter molecule that binds CEA;
contacting the second surface with a second reporter molecule that binds CA125;
contacting the third surface with a third reporter molecule that binds CYFRA21-1;
contacting the fourth surface with a third reporter molecule that binds pro-SFTPB;
measuring the amount of the first reporter molecule associated with the first surface;
measuring the amount of the second reporter molecule associated with the second surface;
measuring the amount of the third reporter molecule associated with the third surface;
measuring the amount of the third reporter molecule associated with the fourth surface;
wherein the amount of the first reporter molecule, the second reporter molecule, the third reporter molecule, and the fourth reporter molecule classifies the subject with indeterminate pulmonary nodules as being at risk of harboring lung cancer or not at risk of harboring lung cancer.

Also provided is a method of distinguishing benign from malignant pulmonary nodules in a subject, comprising:
obtaining a biological sample from the subject;
providing a first surface with means for binding CEA;
providing a second surface with means for binding CA125;
providing a third surface with means for binding CYFRA21-1;
providing a fourth surface with means for binding Pro-SFTPB;
incubating the first surface with the biological sample;
incubating the second surface with the biological sample;
incubating the third surface with the biological sample;
incubating the fourth surface with the biological sample;
contacting the first surface with a first reporter molecule that binds CEA;
contacting the second surface with a second reporter molecule that binds CA125;
contacting the third surface with a third reporter molecule that binds CYFRA21-1;
contacting the fourth surface with a third reporter molecule that binds pro-SFTPB;
measuring the amount of the first reporter molecule associated with the first surface;
measuring the amount of the second reporter molecule associated with the second surface;
measuring the amount of the third reporter molecule associated with the third surface;
measuring the amount of the third reporter molecule associated with the fourth surface;
wherein the amount of the first reporter molecule, the second reporter molecule, the third reporter molecule, and the fourth reporter molecule distinguishes benign pulmonary nodules from malignant pulmonary nodules.

Also provided is a method of determining the risk of a subject with indeterminate pulmonary nodules for harboring lung cancer, comprising:
obtaining a biological sample from the subject;
providing a surface with means for binding CEA, CA125, CYFRA21-1, and Pro-SFTPB;
incubating the surface with the biological sample;
contacting the surface with a first relay molecule that binds CEA;
contacting the surface with a second relay molecule that binds CA125;
contacting the surface with a third relay molecule that binds CYFRA21-1;
contacting the surface with a fourth relay molecule that binds Pro-SFTPB;
contacting the surface with a first reporter molecule that binds to the first relay molecule;
contacting the surface with a second reporter molecule that binds to the second relay molecule;
contacting the surface with a third reporter molecule that binds to the third relay molecule;
contacting the surface with a fourth reporter molecule that binds to the fourth relay molecule;
measuring the amount of the first reporter molecule associated with the first relay molecule and CEA;
measuring the amount of the second reporter molecule associated with the second relay molecule and CA125;
measuring the amount of the third reporter molecule associated with the third relay molecule and CYFRA21-1;
measuring the amount of the fourth reporter molecule associated with the fourth relay molecule and Pro-SFTPB;
wherein the amount of the first reporter molecule, the second reporter molecule, the third reporter molecule, and the fourth reporter molecule classifies the subject with indeterminate pulmonary nodules as being at risk of harboring lung cancer or not at risk of harboring lung cancer.

Also provided is a method of distinguishing benign from malignant pulmonary nodules in a subject, comprising:
obtaining a biological sample from the subject;
providing a surface with means for binding CEA, CA125, CYFRA21-1, and Pro-SFTPB;
incubating the surface with the biological sample;
contacting the surface with a first relay molecule that binds CEA;
contacting the surface with a second relay molecule that binds CA125;
contacting the surface with a third relay molecule that binds CYFRA21-1;
contacting the surface with a fourth relay molecule that binds Pro-SFTPB;
contacting the surface with a first reporter molecule that binds to the first relay molecule;
contacting the surface with a second reporter molecule that binds to the second relay molecule;
contacting the surface with a third reporter molecule that binds to the third relay molecule;
contacting the surface with a fourth reporter molecule that binds to the fourth relay molecule;
measuring the amount of the first reporter molecule associated with the first relay molecule and CEA;
measuring the amount of the second reporter molecule associated with the second relay molecule and CA125;
measuring the amount of the third reporter molecule associated with the third relay molecule and CYFRA21-1;
measuring the amount of the fourth reporter molecule associated with the fourth relay molecule and Pro-SFTPB;
wherein the amount of the first reporter molecule, the second reporter molecule, the third reporter molecule, and the fourth reporter molecule distinguishes benign pulmonary nodules from malignant pulmonary nodules.

Also provided is a method of determining the risk of a subject with indeterminate pulmonary nodules for harboring lung cancer, comprising:
obtaining a biological sample from the subject;
providing a first surface with means for binding CEA;
providing a second surface with means for binding CA125;
providing a third surface with means for binding CYFRA21-1;
providing a fourth surface with means for binding Pro-SFTPB;
incubating the first surface with the biological sample;
incubating the second surface with the biological sample;
incubating the third surface with the biological sample;
incubating the fourth surface with the biological sample;
contacting the first surface with a first relay molecule that binds CEA;
contacting the second surface with a second relay molecule that binds CA125;
contacting the third surface with a third relay molecule that binds CYFRA21-1;
contacting the fourth surface with a fourth relay molecule that binds Pro-SFTPB;
contacting the first surface with a first reporter molecule that binds to the first relay molecule;
contacting the second surface with a second reporter molecule that binds to the second relay molecule;
contacting the third surface with a third reporter molecule that binds to the third relay molecule;
contacting the fourth surface with a fourth reporter molecule that binds to the fourth relay molecule;
measuring the amount of the first reporter molecule that is associated with the first relay molecule and CEA;
measuring the amount of the second reporter molecule that is associated with the second relay molecule and CA125;
measuring the amount of the third reporter molecule that is associated with the third relay molecule and CYFRA21-1;
measuring the amount of the fourth reporter molecule that is associated with the fourth relay molecule and Pro-SFTPB;
wherein the amount of the first reporter molecule, the second reporter molecule, the third reporter molecule, and the fourth reporter molecule classifies the subject with indeterminate pulmonary nodules as being at risk of harboring lung cancer or not at risk of harboring lung cancer.

Also provided is a method of distinguishing benign from malignant pulmonary nodules in a subject, comprising:
obtaining a biological sample from the subject;
providing a first surface with means for binding CEA;
providing a second surface with means for binding CA125;
providing a third surface with means for binding CYFRA21-1;
providing a fourth surface with means for binding Pro-SFTPB;
incubating the first surface with the biological sample;
incubating the second surface with the biological sample;
incubating the third surface with the biological sample;
incubating the fourth surface with the biological sample;
contacting the first surface with a first relay molecule that binds CEA;
contacting the second surface with a second relay molecule that binds CA125;
contacting the third surface with a third relay molecule that binds CYFRA21-1;
contacting the fourth surface with a fourth relay molecule that binds Pro-SFTPB;
contacting the first surface with a first reporter molecule that binds to the first relay molecule;
contacting the second surface with a second reporter molecule that binds to the second relay molecule;
contacting the third surface with a third reporter molecule that binds to the third relay molecule;
contacting the fourth surface with a fourth reporter molecule that binds to the fourth relay molecule;
measuring the amount of the first reporter molecule that is associated with the first relay molecule and CEA;
measuring the amount of the second reporter molecule that is associated with the second relay molecule and CA125;
measuring the amount of the third reporter molecule that is associated with the third relay molecule and CYFRA21-1;
measuring the amount of the fourth reporter molecule that is associated with the fourth relay molecule and Pro-SFTPB;
wherein the amount of the first reporter molecule, the second reporter molecule, the third reporter molecule, and the fourth reporter molecule distinguishes benign pulmonary nodules from malignant pulmonary nodules.

Also provided is a method of determining the risk of a subject with indeterminate pulmonary nodules for harboring lung cancer, comprising:
obtaining a biological sample from the subject;
measuring the level of CEA in the biological sample by contacting the biological sample with a CEA antibody and observing binding between CEA and the antibody;
measuring the level of CA125 in the biological sample by contacting the biological sample with a CA125 antibody and observing binding between CA125 and the antibody;
measuring the level of CYFRA21-1 in the biological sample by contacting the biological sample with a CYFRA21-1 antibody and observing binding between CYFRA21-1 and the antibody;
measuring the level of pro-SFTPB in the biological sample by contacting the biological sample with a pro-SFTPB antibody and observing binding between pro-SFTPB and the antibody;
assigning the condition of the subject with indeterminate pulmonary nodules as either at risk of harboring lung cancer or not at risk of harboring lung cancer, as determined by the measurements of CEA, CA125, CYFRA21-1, and pro-SFTPB levels.

Also provided is a method of distinguishing benign from malignant pulmonary nodules in a subject with indeterminate pulmonary nodules, comprising:
obtaining a biological sample from the subject;
measuring the level of CEA in the biological sample by contacting the biological sample with a CEA antibody and observing binding between CEA and the antibody;
measuring the level of CA125 in the biological sample by contacting the biological sample with a CA125 antibody and observing binding between CA125 and the antibody;
measuring the level of CYFRA21-1 in the biological sample by contacting the biological sample with a CYFRA21-1 antibody and observing binding between CYFRA21-1 and the antibody;
measuring the level of pro-SFTPB in the biological sample by contacting the biological sample with a pro-SFTPB antibody and observing binding between pro-SFTPB and the antibody;
assigning the indeterminate pulmonary nodules as being benign or malignant, as determined by the measurements of CEA, CA125, CYFRA21-1, and pro-SFTPB levels.

Also provided is a method of determining the risk of a subject with indeterminate pulmonary nodules for harboring lung cancer, comprising:
obtaining a biological sample from the subject;
measuring the level of CEA in the biological sample;
measuring the level of CA125 in the biological sample;
measuring the level of CYFRA21-1 in the biological sample;
measuring the level of pro-SFTPB in the biological sample;
determining the level of CEA relative to a first standard value, wherein the ratio is predictive of presence of lung cancer;
determining the level of CA125 relative to a second standard value, wherein the ratio is predictive of presence of lung cancer;
determining the level of CYFRA21-1 relative to a third standard value, wherein the ratio is predictive of presence of lung cancer; and
determining the level of pro-SFTPB relative to a fourth standard value, wherein the ratio is predictive of presence of lung cancer; and
assigning the condition of the subject with indeterminate pulmonary nodules as either at risk of harboring lung cancer or not at risk of harboring lung cancer, as determined by statistical analysis of the ratios of CEA, CA125, CYFRA21-1, and pro-SFTPB levels.

Also provided is a method of distinguishing benign from malignant pulmonary nodules in a subject with indeterminate pulmonary nodules, comprising:
obtaining a biological sample from the subject;
measuring the level of CEA in the biological sample;
measuring the level of CA125 in the biological sample;
measuring the level of CYFRA21-1 in the biological sample;
measuring the level of pro-SFTPB in the biological sample;
determining the level of CEA relative to a first standard value, wherein the ratio is predictive of presence of malignant pulmonary nodules;
determining the level of CA125 relative to a second standard value, wherein the ratio is predictive of presence of malignant pulmonary nodules;
determining the level of CYFRA21-1 relative to a third standard value, wherein the ratio is predictive of presence of malignant pulmonary nodules; and
determining the level of pro-SFTPB relative to a fourth standard value, wherein the ratio is predictive of presence of malignant pulmonary nodules; and
assigning the pulmonary nodules as being benign or malignant as determined by statistical analysis of the ratios of CEA, CA125, CYFRA21-1, and pro-SFTPB levels.

Also provided is a method of distinguishing benign from malignant pulmonary nodules in a subject with indeterminate pulmonary nodules, comprising:
obtaining a biological sample from the subject;

measuring the levels of the CEA, CA125, CYFRA21-1, and pro-SFTPB biomarkers in the biological sample; and

calculating a predictive factor as determined by statistical analysis of the CEA, CA125, CYFRA21-1, and pro-SFTPB levels.

Also provided is a method of predicting the risk of a subject with indeterminate pulmonary nodules for harboring lung, comprising:
obtaining a biological sample from the subject;
measuring the levels of the CEA, CA125, CYFRA21-1, and pro-SFTPB biomarkers in the biological sample; and
calculating a predictive factor as determined by statistical analysis of the CEA,
CA125, CYFRA21-1, and pro-SFTPB levels.

Also provided is a method of determining the risk of a subject with indeterminate pulmonary nodules for harboring lung cancer, comprising:
obtaining a biological sample from the subject;
measuring the levels of CEA, CA125, CYFRA21-1, and pro-SFTPB biomarkers in the biological sample;
assigning the condition of the subject with indeterminate pulmonary nodules as either at risk of harboring lung cancer or not at risk of harboring lung cancer, as determined by statistical analysis of the levels of CEA, CA125, CYFRA21-1, and pro-SFTPB in the biological sample.

Also provided is a method of distinguishing benign from malignant pulmonary nodules in a subject with indeterminate pulmonary nodules, comprising:
obtaining a biological sample from the subject;
measuring the levels of CEA, CA125, CYFRA21-1, and pro-SFTPB biomarkers in the biological sample;
assigning the indeterminate pulmonary nodules as being benign or malignant, as determined by statistical analysis of the levels of CEA, CA125, CYFRA21-1, and pro-SFTPB in the biological sample.

Also provided is a method for determining the risk of a subject with indeterminate pulmonary nodules for harboring lung cancer using a biological sample obtained from a subject suspected of having lung cancer, comprising:
assaying for the level of CEA present in the biological sample using at least one antibody or antibody fraction specific for CEA; and
assaying for the level of CA125 present in the biological sample using at least one antibody or antibody fraction specific for CA125; and
assaying for the level of CYFRA21-1 present in the biological sample using at least one antibody or antibody fraction specific for CYFRA21-1; and
assaying for the level of pro-SFTPB present in the biological sample using at least one antibody or antibody fraction specific for pro-SFTPB; and
determining whether the levels of CEA, CA125, CYFRA21-1, and pro-SFTPB are indicative of the subject having lung cancer.

Also provided is a method for distinguishing benign from malignant pulmonary nodules in a subject with indeterminate pulmonary nodules using a biological sample obtained from a subject suspected of having lung cancer, comprising:
assaying for the level of CEA present in the biological sample using at least one antibody or antibody fraction specific for CEA; and
assaying for the level of CA125 present in the biological sample using at least one antibody or antibody fraction specific for CA125; and
assaying for the level of CYFRA21-1 present in the biological sample using at least one antibody or antibody fraction specific for CYFRA21-1; and
assaying for the level of pro-SFTPB present in the biological sample using at least one antibody or antibody fraction specific for pro-SFTPB; and
determining whether the levels of CEA, CA125, CYFRA21-1, and pro-SFTPB are indicative of the indeterminate pulmonary nodules being benign or malignant.

Also provided is a method for determining the risk of a subject with indeterminate pulmonary nodules for harboring lung cancer comprising:
obtaining a biological sample sample from a subject;
performing an immunoassay on the sample with an anti-CEA antibody or antigen-binding fragment thereof;
performing an immunoassay on the sample with an anti-CA125 antibody or antigen-binding fragment thereof;
performing an immunoassay on the sample with an anti-CYFRA21-1 antibody or antigen-binding fragment thereof;
performing an immunoassay on the sample with an anti-pro-SFTPB antibody or antigen-binding fragment thereof;
wherein binding of the antibodies is indicative of lung cancer in the subject and the immunoassay can detect early stage lung cancer.

Also provided is a method for distinguishing benign from malignant pulmonary nodules in a subject with indeterminate pulmonary nodules comprising:
obtaining a biological sample sample from a subject;
performing an immunoassay on the sample with an anti-CEA antibody or antigen-binding fragment thereof;
performing an immunoassay on the sample with an anti-CA125 antibody or antigen-binding fragment thereof;
performing an immunoassay on the sample with an anti-CYFRA21-1 antibody or antigen-binding fragment thereof;
performing an immunoassay on the sample with an anti-pro-SFTPB antibody or antigen-binding fragment thereof;
wherein binding of the antibodies is indicative of malignant pulmonary nodules in the subject and the immunoassay.

Also provided is a method for determining the risk of a subject with indeterminate pulmonary nodules for harboring lung cancer comprising:
obtaining a biological sample sample from the subject;
performing an immunoassay with an anti-CEA antibody or antigen-binding fragment thereof;
performing an immunoassay with an anti-CA125 antibody or antigen-binding fragment thereof;
performing an immunoassay with an anti-CYFRA21-1 antibody or antigen-binding fragment thereof;
performing an immunoassay with an anti-pro-SFTPB antibody or antigen-binding fragment thereof;
determining whether the levels of CEA, CA125, CYFRA21-1, and pro-SFTPB are indicative of the subject having lung cancer.

Also provided is a method for distinguishing benign from malignant pulmonary nodules in a subject with indeterminate pulmonary nodules comprising:
obtaining a biological sample sample from the subject;
performing an immunoassay with an anti-CEA antibody or antigen-binding fragment thereof;
performing an immunoassay with an anti-CA125 antibody or antigen-binding fragment thereof;
performing an immunoassay with an anti-CYFRA21-1 antibody or antigen-binding fragment thereof;
performing an immunoassay with an anti-pro-SFTPB antibody or antigen-binding fragment thereof;
determining whether the levels of CEA, CA125, CYFRA21-1, and pro-SFTPB are indicative of the indeterminate pulmonary nodules being benign or malignant.

Also provided is a method of determining the risk of a subject with indeterminate pulmonary nodules for harboring lung cancer, comprising:
obtaining a biological sample from the patient;
measuring the level of diacetylspermine (DAS) in the biological sample;
wherein the amount of diacetylspermine (DAS) classifies the subject with indeterminate pulmonary nodules as being at risk of harboring lung cancer or not at risk of harboring lung cancer.

Also provided is a method of distinguishing benign from malignant pulmonary nodules in a subject with indeterminate pulmonary nodules, comprising:
obtaining a biological sample from the patient;
measuring the level of diacetylspermine (DAS) in the biological sample;
wherein the amount of diacetylspermine (DAS) classifies the indeterminate pulmonary nodules as being benign or malignant.

Also provided is a method of determining the risk of a subject with indeterminate pulmonary nodules for harboring lung cancer, comprising a plasma-derived biomarker panel and a protein marker panel:
wherein the plasma-derived biomarker panel comprises diacetylspermine (DAS);
wherein the protein biomarker panel comprises CEA, CA125, CYFRA21-1, and pro-SFTPB;
wherein the method comprises:
   obtaining a biological sample from the subject;
   measuring the levels of the plasma-derived biomarkers and the protein biomarkers in the biological sample;
wherein the amount of the plasma-derived biomarkers and the protein biomarkers classifies the subject as being at risk of with indeterminate pulmonary nodules harboring lung cancer or not at risk of harboring lung cancer.

Also provided is a method of distinguishing benign from malignant pulmonary nodules in a subject with indeterminate pulmonary nodules from a plasma-derived biomarker panel and a protein marker panel:
wherein the plasma-derived biomarker panel comprises diacetylspermine (DAS);
wherein the protein biomarker panel comprises CEA, CA125, CYFRA21-1, and pro-SFTPB;
wherein the method comprises:
   obtaining a biological sample from the subject;
   measuring the levels of the plasma-derived biomarkers and the protein biomarkers in the biological sample;
   wherein the amount of the plasma-derived biomarkers and the protein biomarkers classifies the indeterminate pulmonary nodules as being benign or malignant.

Also provided is a method of determining the risk of a subject with indeterminate pulmonary nodules for harboring lung cancer, comprising determining the levels of one or more protein biomarkers and one or more metabolite markers, said method comprising:
obtaining a biological sample from the subject;
contacting the sample with a first reporter molecule that binds CEA antigen;
contacting the sample with a second reporter molecule that binds CA125 antigen;
contacting the sample with a third reporter molecule that binds CYFRA21-1 antigen; and
contacting the sample with a fourth reporter molecule that binds pro-SFTPB antigen; and
determining the levels of the one or more biomarkers, wherein the one or more biomarkers is selected from the group consisting of diacetylspermine (DAS);
wherein the amount of the first reporter molecule, the second reporter molecule, the third reporter molecule, the fourth reporter molecule, and the one or more biomarkers classifies the subject with indeterminate pulmonary nodules as being at risk of harboring lung cancer or not at risk of harboring lung cancer.

Also provided is a method of distinguishing benign from malignant pulmonary nodules in a subject with indeterminate pulmonary nodules, comprising determining the levels of one or more protein biomarkers and one or more metabolite markers, said method comprising:
obtaining a biological sample from the subject;
contacting the sample with a first reporter molecule that binds CEA antigen;
contacting the sample with a second reporter molecule that binds CA125 antigen;
contacting the sample with a third reporter molecule that binds CYFRA21-1 antigen; and
contacting the sample with a fourth reporter molecule that binds pro-SFTPB antigen; and
determining the levels of the one or more biomarkers, wherein the one or more biomarkers is selected from the group consisting of diacetylspermine (DAS);
wherein the amount of the first reporter molecule, the second reporter molecule, the third reporter molecule, the fourth reporter molecule, and the one or more biomarkers classifies the indeterminate pulmonary nodules as being benign or malignant.

Also provided is a method of determining the risk of a subject with indeterminate pulmonary nodules for harboring lung cancer, comprising:
obtaining a biological sample from the subject;
measuring the levels of CEA, CA125, CYFRA21-1, and pro-SFTPB antigens in the biological sample; and
measuring the levels of one or more metabolite markers selected from the group consisting of diacetylspermine (DAS) in the biological sample;
assigning the condition of the subject with indeterminate pulmonary nodules as either at risk of harboring lung cancer or not at risk of harboring lung cancer, as determined by statistical analysis of the levels of CEA antigen, CA125 antigen, CYFRA21-1 antigen, pro-SFTPB antigen, and diacetylspermine (DAS) in the biological sample.

Also provided is a method for distinguishing benign from malignant pulmonary nodules in a subject, comprising:
obtaining a biological sample from the subject;
measuring the levels of CEA, CA125, CYFRA21-1, and pro-SFTPB antigens in the biological sample; and
measuring the levels of one or more metabolite markers selected from the group consisting of diacetylspermine (DAS) in the biological sample;
assigning the condition of the modules as either benign or malignant, as determined by statistical analysis of the levels of CEA antigen, CA125 antigen, CYFRA21-1 antigen, pro-SFTPB antigen, and diacetylspermine (DAS) in the biological sample.

In some embodiments, the method further comprises:
measuring the level of diacetylspermine (DAS) in the biological sample;
   wherein the amount of diacetylspermine (DAS) classifies the patient as being at risk of harboring lung cancer or not at risk of harboring lung cancer or distinguishes the patient as having benign or malignant pulmonary nodules. In some embodiments, the method further comprises measuring the levels of miR-320, miR-210, and/or miR-21 in the biological sample;
   wherein the amount of miR-320, miR-210, and/or miR-21 classifies the patient as being at risk of harboring lung cancer or not at risk of harboring lung cancer or distinguishes the patient as having benign or malignant pulmonary nodules.

In some embodiments, the method further comprises measuring the levels of diacetylspermine (DAS), diacetyspermidine, acetylspermidine, 1-methyladenosine, n-acetyllactosamine, arginine, and dimethyl-argininein the biological sample;
wherein the amount of diacetylspermine (DAS), diacetyspermidine, acetylspermidine, 1-methyladenosine, n-acetyllactosamine, arginine, and dimethyl-arginineclassifies the patient as being at risk of harboring lung cancer or not at risk of harboring lung cancer or distinguishes the patient as having benign or malignant pulmonary nodules.

In some embodiments, the method further comprises measuring the levels of miR-320, miR-210, and/or miR-21 in the biological sample; and
measuring the levels of diacetylspermine (DAS), diacetyspermidine, acetylspermidine, 1-methyladenosine, n-acetyllactosamine, arginine, and dimethyl-arginine in the biological sample;
wherein the amount of miR-320, miR-210, and/or miR-21, and the amount of diacetylspermine (DAS), diacetyspermidine, acetylspermidine, 1-methyladenosine, n-acetyllactosamine, arginine, and dimethyl-arginine classifies the patient as being at risk of harboring lung cancer or not at risk of harboring lung cancer or distinguishes the patient as having benign or malignant pulmonary nodules.

In some embodiments, the subject is determined to have lung cancer based on the measured concentrations of the biomarkers.

In some embodiments, the method further comprises: comparing the measured concentrations of each biomarker in the biological sample to the prediction of a statistical model.

In some embodiments, the method further comprises administering at least one alternate diagnostic test for a subject assigned as having lung cancer.

In some embodiments, the at least one alternate diagnostic test comprises an assay or sequencing of at least one ctDNA.

In some embodiments, the lung cancer is diagnosed at or before the borderline resectable stage.

In some embodiments, the lung cancer is diagnosed at the resectable stage.

In some embodiments, the reference subject or group is healthy.

In some embodiments, the markers consist of CEA, CA125, CYFRA21-1, Pro-SFTPB, and diacetylspermine (DAS).

In some embodiments, the markers consist of miRNA-320 and miRNA-210.

In some embodiments, the markers consist of diacetylspermine (DAS), diacetyspermidine, acetylspermidine, 1-methyladenosine, n-acetyllactosamine, arginine, and dimethyl-arginine.

In some embodiments, the markers consist of miRNA-320, miRNA-210, diacetylspermine (DAS), diacetyspermidine, acetylspermidine, 1-methyladenosine, n-acetyllactosamine, arginine, and dimethyl-arginine.

In some embodiments, the panel is selected from the group consisting of:
a. the panel consisting of CEA, CA125, CYFRA21-1, and Pro-SFTPB; or
b. the panel consisting of CEA, CA125, CYFRA21-1, Pro-SFTPB, and diacetylspermine (DAS).

In some embodiments, the panel is selected from the group consisting of:
a. the panel consisting of CEA, CA125, CYFRA21-1, and Pro-SFTPB; or
b. the panel consisting of CEA, CA125, CYFRA21-1, Pro-SFTPB, and diacetylspermine (DAS).
c. the panel consisting of miRNA-320 and miRNA-210; or
d. the panel consisting of diacetylspermine (DAS), diacetyspermidine, acetylspermidine, 1-methyladenosine, n-acetyllactosamine, arginine, and dimethyl-arginine; or
e. any combination of a-d. In some embodiments, the amounts of CEA, CA125, CYFRA21-1, and pro-SFTPB or the reporter molecules bound thereto are elevated in the subject relative to a healthy subject.

In some embodiments, the levels of CEA, CA125, CYFRA21-1, and pro-SFTPB are elevated in the subject relative to a healthy subject.

In some embodiments, the levels of miR-320 and miR-210 are reduced in the subject relative to a healthy subject.

In some embodiments, the levels of diacetylspermine (DAS), diacetyspermidine, acetylspermidine, 1-methyladenosine, n-acetyllactosamine, arginine, and dimethyl-arginine are elevated in the subject relative to a healthy subject.

In some embodiments, the amount of CEA, CA125, CYFRA21-1, and pro-SFTPB is quantified.

In some embodiments, the amount of miR-320 and miR-210 is quantified.

In some embodiments, the amount of diacetylspermine (DAS), diacetyspermidine, acetylspermidine, 1-methyladenosine, n-acetyllactosamine, arginine, and dimethyl-arginine is quantified.

In some embodiments, the concentrations of CEA, CA125, CYFRA21-1, Pro-SFTPB, and diacetylspermine (DAS) are measured.

In some embodiments, the concentrations of miR-320 and miR-210 are measured.

In some embodiments, the concentrations of diacetylspermine (DAS), diacetyspermidine, acetylspermidine, 1-methyladenosine, n-acetyllactosamine, arginine, and dimethyl-arginine are measured.

In some embodiments, at least one of the surfaces further comprises at least one reporter molecule that selectively binds to a biomarker or antigen selected from CEA, CA125, CYFRA21-1, and Pro-SFTPB.

In some embodiments, the first reporter binds selectively to CEA.

In some embodiments, the second reporter binds selectively to CA125.

In some embodiments, the third reporter binds selectively to CYFRA21-1.

In some embodiments, the fourth reporter binds selectively to Pro-SFTPB.

In some embodiments, determination of CEA, CA125, CYFRA21-1, and pro-SFTPB levels is made at substantially the same time.

In some embodiments, determination of CEA, CA125, CYFRA21-1, and pro-SFTPB levels is made in a stepwise manner.

In some embodiments, the method further comprises inclusion of subject history information into the assignment of having lung cancer or not having lung cancer.

In some embodiments, at least one of the surfaces further comprises at least one receptor molecule that selectively binds to a biomarker selected from CEA, CA125, CYFRA21-1, and Pro-SFTPB.

In some embodiments, the amounts of CEA antigen, CA125 antigen, CYFRA21-1 antigen, and pro-SFTPB antigen are elevated in comparison to the levels of CEA antigen, CA125 antigen, CYFRA21-1 antigen, and pro-SFTPB antigen in a reference subject or group that does not have lung cancer.

In some embodiments, the levels of CEA antigen, CA125 antigen, CYFRA21-1 antigen, and pro-SFTPB antigen are elevated in comparison to the levels of CEA antigen, CA125 antigen, CYFRA21-1 antigen, and pro-SFTPB antigen in a reference subject or group that has adenocarcinoma.

In some embodiments, the levels of CEA antigen, CA125 antigen, CYFRA21-1 antigen, and pro-SFTPB antigen are elevated in comparison to the levels of CEA antigen, CA125 antigen, CYFRA21-1 antigen, and pro-SFTPB antigen in a reference subject or group that has squamous cell cancer.

In some embodiments, the sample comprises a biological sample selected from blood, plasma, and serum. In some embodiments, the biological sample is serum.

In some embodiments, detection of the amount of CEA, CA125, CYFRA21-1, pro-SFTPB, and diacetylspermine (DAS) comprises the use of a solid particle.

In some embodiments, at least one of the surfaces is the surface of a solid particle.

In some embodiments, the solid particle is a bead.

In some embodiments, at least one of the reporter molecules is linked to an enzyme.

In some embodiments, at least one of the reporter molecules provides a detectable signal.

In some embodiments, the detectable signal is detectable by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In some embodiments, the spectrometric method is mass spectrometry.

In some embodiments, the panel comprises biomarkers that have been identified by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In some embodiments, the panel comprises biomarkers that have been identified by UV-visible spectroscopy or proton NMR spectroscopy.

In some embodiments, the method further comprises comparing the amount of CEA, CA125, CYFRA21-1, and pro-SFTPB with a cutoff value comprises an AUC (95% CI) of at least 0.83. In some embodiments, the cutoff value comprises an AUC (95% CI) of at least 0.80. In some embodiments, the cutoff value comprises an AUC (95% CI) of at least 0.81. In some embodiments, the cutoff value comprises an AUC (95% CI) of at least 0.88. In some embodiments, the classification of the subject as having lung cancer has a sensitivity of 73% at 90% specificity, 62% at 95% specificity, and/or 42% at 99% specificity. In some embodiments, the classification of the subject as having lung cancer has a sensitivity of 73% at 90% specificity. In some embodiments, the classification of the subject as having lung cancer has a sensitivity of 62% at 95% specificity. In some embodiments, the classification of the subject as having lung cancer has a sensitivity of 42% at 99% specificity. In some embodiments, the classification of the subject as having lung cancer has an increase in sensitivity of 11% at 95% specificity compared to control. In some embodiments, the classification of the subject as having lung cancer has an increase in the AUC of 7% compared to control.

Also provided is a method comprising:
a) obtaining a sample from a subject with pulmonary nodules;
b) measuring a panel of markers in the sample, wherein the markers comprise CEA, CA125, Cyfra 21-1, and diacetylspermine (DAS);
c) determining a biomarker score for each marker;
d) summing the biomarker scores for each marker to obtain a composite score for each subject, quantifying the increased risk for the presence of lung cancer for the subject as a risk score, wherein the composite score is matched to a risk category of a grouping of stratified subject populations, wherein each risk category comprises a multiplier indicating increased likelihood of having the lung cancer correlated to a range of composite scores as compared to use of a single threshold value, wherein the multiplier is determined from positive predictive scores of retrospective samples; and,
e) administering a computerized tomography (CT) scan or other imagine modality to the subject with a quantified increased risk for the presence of lung cancer.

Also provided is a method comprising:
a) obtaining a sample from a subject with pulmonary nodules;
b) measuring a panel of markers in the sample, wherein the markers comprise CEA, CA125, Cyfra 21-1, and diacetylspermine (DAS); and/or miRNA-320 and miRNA-210; and/or diacetylspermine, diacetyspermidine, acetylspermidine, 1-methyladenosine, n-acetyllactosamine, arginine, and dimethyl-arginine; c) determining a biomarker score for each marker;
d) summing the biomarker scores for each marker to obtain a composite score for each subject, quantifying the increased risk for the presence of lung cancer for the subject as a risk score, wherein the composite score is matched to a risk category of a grouping of stratified subject populations, wherein each risk category comprises a multiplier indicating increased likelihood of having the lung cancer correlated to a range of composite scores as compared to use of a single threshold value, wherein the multiplier is determined from positive predictive scores of retrospective samples; and,
e) administering a computerized tomography (CT) scan or other imagine modality to the subject with a quantified increased risk for the presence of lung cancer.

In some embodiments, the grouping of a stratified subject population, the multiplier indicating increased likelihood of having the cancer and the range of composite scores are determined from retrospective clinical samples of a population.

In some embodiments, the risk category further comprises a risk identifier.

In some embodiments, the risk identifier is selected from low risk, intermediate-low risk, intermediate risk, intermediate-high risk and highest risk.

In some embodiments, calculating the multiplier indicating increased likelihood of having the cancer for each risk category comprises stratifying the subject cohort based on retrospective biomarker scores and weighting a known prevalence of the cancer in the cohort by a positive predictive score for each stratified population.

In some embodiments, the grouping of a stratified subject population comprises at least three risk categories wherein the multiplier indicating increased likelihood of having cancer is about 2 or greater.

In some embodiments, the grouping of a stratified subject population comprises at least two risk categories wherein the multiplier indicating increased likelihood of having cancer is about 5 or greater.

In some embodiments, the subject is aged 50 years or older and has a history of smoking tobacco.

In some embodiments, the method further comprises generating a risk categorization table, wherein the panel of markers is measured, a biomarker score for each marker is determined, a composite score is obtained by summing the biomarker scores; determining a threshold value used to divide the composite scores into risk groups and assigning a multiplier to each group indicating the likelihood of an asymptomatic subject having a quantified increased risk for the presence of cancer.

In some embodiments, the groups are in a form selected from an electronic table form, a software application, a computer program, and an excel spreadsheet.

In some embodiments, the panel of markers comprise proteins, polypeptides, or metabolites measured in a binding assay.

In some embodiments, the panel of markers comprise proteins or polypeptides measured using a flow cytometer.

Also provided is a method of treating a subject with indeterminate pulmonary nodules suspected of harboring lung cancer, comprising:
analyzing the subject with indeterminate pulmonary nodules for risk of harboring lung cancer with a method described herein; and
administering a therapeutically effective amount of a treatment for the cancer.

Also provided is a method of treating a subject with indeterminate pulmonary nodules suspected of harboring lung cancer, comprising:
analyzing the subject with indeterminate pulmonary nodules for risk of harboring lung cancer with a method described herein; and
administering a therapeutically effective amount of a treatment for the cancer if the indeterminate pulmonary nodules are malignant.

Also provided is a method of treatment or prevention of progression of lung cancer in a subject with indeterminate pulmonary nodules in whom the levels of CEA antigen, CA125 antigen, CYFRA21-1 antigen, and pro-SFTPB antigen classifies the subject with indeterminate pulmonary nodules as having or being at risk of harboring lung cancer comprising one or more of:
i. administering a chemotherapeutic drug to the subject with lung cancer;
ii. administering therapeutic radiation to the subject with lung cancer; and
iii. surgery for partial or complete surgical removal of cancerous tissue in the subject with lung cancer.

Also provided is a method of treatment or prevention of progression of lung cancer in a subject with indeterminate pulmonary nodules in whom the levels of CEA antigen, CA125 antigen, CYFRA21-1 antigen, pro-SFTPB antigen, diacetylspermine (DAS) classifies the subject with indeterminate pulmonary nodules as having or being at risk of harboring lung cancer comprising one or more of:
i) administering a chemotherapeutic drug to the subject with lung cancer;
ii) administering therapeutic radiation to the subject with lung cancer; and
iii) surgery for partial or complete surgical removal of cancerous tissue in the subject with lung cancer.

Also provided is a method for detecting and treating lung cancer, comprising:
detecting CEA, CA125, CYFRA21-1, and pro-SFTPB, in a biological sample obtained from a human with indeterminate pulmonary nodules, via an immunoassay;
quantifying the amounts CEA, CA125, CYFRA21-1, and pro-SFTPB in said collected sample;
comparing the amounts of CEA, CA125, CYFRA21-1, and pro-SFTPB with a cutoff value to determine whether said human is at increased risk of having lung cancer or not;
wherein if the levels are above the cutoff value said human has lung cancer, and administering a treatment for lung cancer to said human having lung cancer.

Also provided is a method for detecting and treating lung cancer, comprising:
detecting CEA, CA125, CYFRA21-1, and pro-SFTPB, in a biological sample obtained from a human with indeterminate pulmonary nodules, via an immunoassay; and/or
detecting miRNA-320 and miRNA-210, in a biological sample obtained from a human with indeterminate pulmonary nodules, via an immunoassay; and/or
detecting diacetylspermine (DAS), diacetyspermidine, acetylspermidine, 1-methyladenosine, n-acetyllactosamine, arginine, and dimethyl-arginine, in a biological sample obtained from a human with indeterminate pulmonary nodules, via an immunoassay; and/or
quantifying the amounts of miRNA-320 and miRNA-210 in said collected sample; and/or
quantifying the amounts of diacetylspermine (DAS), diacetyspermidine, acetylspermidine, 1-methyladenosine, n-acetyllactosamine, arginine, and dimethyl-arginine in said collected sample;
quantifying the amounts CEA, CA125, CYFRA21-1, and pro-SFTPB in said collected sample;
comparing the amounts of CEA, CA125, CYFRA21-1, and pro-SFTPB; and/or the amounts of miRNA-320 and miRNA-210; and/or the amounts of diacetylspermine (DAS), diacetyspermidine, acetylspermidine, 1-methyladenosine, n-acetyllactosamine, arginine, and dimethyl-arginine with a cutoff value to determine whether said human is at increased risk of having lung cancer or not; wherein if the levels are above the cutoff value said human has lung cancer, and administering a treatment for lung cancer to said human having lung cancer.

Also provided is a method of treating a subject with indeterminate pulmonary nodules suspected of harboring lung cancer, comprising:
analyzing the subject with indeterminate pulmonary nodules for risk of harboring lung cancer or for having malignant pulmonary nodules with a method described herein; and
administering a therapeutically effective amount of a treatment for the cancer.

Also provided is a kit for any of the methods described herein, comprising:
a reagent solution that comprises
a first solute for detection of CEA;
a second solute for detection of CA125;
a third solute for detection of CYFRA21-1; and
a fourth solute for detection of pro-SFTPB.

Also provided is a kit for any of the methods described herein, comprising:
a reagent solution that comprises
   a first solute for detection of CEA antigen;
   a second solute for detection of CA125 antigen;
   a third solute for detection of CYFRA21-1 antigen;
   a fourth solute for detection of pro-SFTPB antigen; and
   a fifth solute for detection of diacetylspermine (DAS).

Also provided is a kit for any of the methods described herein, comprising:
a first reagent solution that comprises a first solute for detection of CEA;
a second reagent solution that comprises a second solute for detection of CA125;
a third reagent solution that comprises a third solute for detection of CYFRA21-1;
and
a fourth reagent solution that comprises a fourth solute for detection of pro-SFTPB.

Also provided is a kit for any of the methods described herein, comprising:
a first reagent solution that comprises a first solute for detection of CEA antigen;
a second reagent solution that comprises a second solute for detection of CA125 antigen;
a third reagent solution that comprises a third solute for detection of CYFRA21-1 antigen;
a fourth reagent solution that comprises a fourth solute for detection of pro-SFTPB;
a fifth reagent solution that comprises a fifth solute for detection of diacetylspermine (DAS).

Also provided is a kit for determining the presence of indicators of lung cancer in a sample from a subject with indeterminate pulmonary nodules comprising:
(a) antigen-binding reagents that bind to each of the protein biomarkers selected from the group consisting of CEA, CA125, CYFRA21-1, and pro-SFTPB, or an array comprising said antigen-binding reagents; and
(b) instructions for performing a method for determining the presence of lung cancer in an individual.

In some embodiments, the kit further comprises a device for contacting the reagent solutions with a biological sample.

In some embodiments, the kit further comprises at least one surface with means for binding at least one biomarker or antigen.

In some embodiments, the at least one biomarker is selected from the group consisting of CEA, CA125, CYFRA21-1, and pro-SFTPB.

In some embodiments, the at least one surface comprises a means for binding ctDNA.

In some embodiments, the kit further comprises an antibody or antigen-binding fragment thereof that binds to the metabolite biomarker diacetylspermine (DAS).

In some embodiments, the antigen-binding reagent comprises antibodies or antigen-binding fragments thereof, RNA, DNA, or RNA/DNA hybrids.

The foregoing has outlined rather broadly the features and technical benefits of the disclosure in order that the detailed description may be better understood. It should be appreciated by those skilled in the art that the specific embodiments disclosed may be readily utilized as a basis for modifying or designing other structures or processes for carrying out the same purposes of the disclosure. It is to be understood that the present disclosure is not limited to the particular embodiments described, as variations of the particular embodiments may be made and still fall within the scope of the appended claims.

### Definitions

As used herein, the term “4MP” refers to a 4-protein marker panel which includes the proprotein form of surfactant protein B (pro-SFTPB) and three other markers with known utility in diagnosing lung cancer: cancer antigen 125 (CA125), cytokeratin-19 fragment (CYFRA 21-1), and carcinoembryonic antigen (CEA). Additional details about the 4MP are described in WO 2018/148600, which is incorporated herein by reference for all purposes.

As used herein, the term “2-miRNA panel” refers to a panel of 2 miRNAs, which includes miR-320 and miR-210. In some embodiments, the 2-miRNA panel may be combined with the 4MP to enhance detection of lung cancer in biological samples from patients suspected as having or developing lung cancer.

As used herein, the term “3-miRNA panel” refers to a panel of 3 miRNAs, which includes miR-320, miR-210, and miR-21. In some embodiments, the 3-miRNA panel may be combined with the 4MP to enhance detection of lung cancer in biological samples from patients suspected as having or developing lung cancer.

As used herein, the term “7-metabolite panel” refers to a panel of 7 cancer-associated metabolites, which include diacetylspermine, diacetyspermidine, acetylspermidine, 1-methyladenosine, n-acetyllactosamine, arginine, and dimethyl-arginine. In some embodiments, the 7-metabolite panel may be combined with the 4MP to enhance detection of lung cancer in biological samples from patients suspected as having or developing lung cancer.

In some embodiments, the 2-miRNA panel and the 7-metabolite panel may both be combined with the 4MP to enhance detection of lung cancer in biological samples of patients suspected as having lung cancer. In some embodiments, the 3-miRNA panel and the 7-metabolite panel may both be combined with the 4MP to enhance detection of lung cancer in biological samples of patients suspected as having lung cancer. In some embodiments, combination of the 2-miRNA panel or the 7-metabolite panel, or both the 2-miRNA panel and the 7-metabolite panel, with the 4MP results in an increase in the sensitivity and/or specificity of the diagnostic test, or an increase in the prognostic value of the combination of markers or panels, when compared to the 4MP alone. In some embodiments, combination of the 3-miRNA panel or the 7-metabolite panel, or both the 3-miRNA panel and the 7-metabolite panel, with the 4MP results in an increase in the sensitivity and/or specificity of the diagnostic test, or an increase in the prognostic value of the combination of markers or panels, when compared to the 4MP alone.

As used herein, the term “lung tissue” refers to tissue of the lungs themselves, as well as the tissue adjacent to and/or within the strata underlying the lungs and supporting structures such as the pleura, intercostal muscles, ribs, and other elements of the respiratory system. The respiratory system itself is taken in this context as representing nasal cavity, sinuses, pharynx, larynx, trachea, bronchi, lungs, lung lobes, aveoli, aveolar ducts, aveolar sacs, aveolar capillaries, bronchioles, respiratory bronchioles, visceral pleura, parietal pleura, pleural cavity, diaphragm, epiglottis, adenoids, tonsils, mouth and tongue, and the like.

As used herein, the term “lung cancer” refers to a malignant neoplasm of the lung characterized by the abnormal proliferation of cells, the growth of which cells exceeds and is uncoordinated with that of the normal tissues around it. The American Lung Cancer Society provides the following lung cancer staging definitions. In stage T0, there is no evidence of primary tumor. In stage Tis, there is carcinoma in situ. Stage T1 denotes tumors of 3 cm or less. Stage T1a denotes tumors having 2 cm or less. Stage T1b denotes a tumor having a dimension of more than 2 cm but less than 3 cm. Stage T2 denotes tumors of having dimensions of more than 3 cm but 7 cm or less. Stage T2a denotes tumors having dimensions of more than 3 cm but 5 cm or less. Stage T2b denotes tumors having more than 5 cm in dimension but being 7 cm or less. Stage T3 denotes tumors that are more than 7 cm or those tumors that invades the chest wall, phrenic nerve, diaphragm, parietal pleura, parietal pericardium or mediastinal pleura; or a tumor in the main bronchus that is less than 2 cm. Stage T4 denotes tumors that invades any of: heart, esophagus, mediastinum, trachea, recurrent laryngeal nerve, carina, vertebral body, or a separate tumor nodule in a different ipsilateral lobe.

As used herein, the term “lung cancer-positive” refers to classification of a subject as having lung cancer.

As used herein, the term “lung cancer-negative” refers to classification of a subject as not having lung cancer.

As used herein, the term “pulmonary nodules” (PNs) refers to lung lesions that can be visualized by radiographic techniques. A pulmonary nodule is any nodule less than or equal to three centimeters in diameter. In some embodiments, a pulmonary nodule has a diameter of about 0.8 cm to 2 cm.

As used herein, the term “masses” or “pulmonary masses” refers to lung nodules that are greater than three centimeters maximal diameter.

As used herein, the terms “subject” or “patient” refer to a mammal, preferably a human, for whom a classification as lung cancer-positive or lung cancer-negative is desired, and for whom further treatment can be provided.

As used herein, a “reference patient,” “reference subject,” or “reference group” refers to a group of patients or subjects to which a test sample from a patient or subject suspected of having or being at risk of harboring lung cancer may be compared. In some embodiments, such a comparison may be used to determine whether the test subject has lung cancer. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual in whom no evidence of lung cancer is found, i.e., the individual does not have lung cancer. Such an individual may be classified as “lung cancer-negative” or as having healthy lungs, or normal, non-compromised lung function. A healthy patient or subject has no symptoms of lung cancer or other lung disease. In some embodiments, a healthy patient or subject may be used as a reference patient for comparison to diseased or suspected diseased samples for determination of lung cancer in a patient or a group of patients.

As used herein, the terms “treatment” or “treating” refer to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as Erlotinib (TARCEVA and others), Afatinib (GILOTRIF), Gefitinib (IRESSA), Bevacizumab (AVASTIN), Crizotinib (XALKORI), Ceritinib (ZYKADIA). cisplatin (PLATINOL), carboplatin (PARAPLATIN), docetaxel (TAXOTERE), gemcitabine (GEMZAR), paclitaxel (TAXOL and others), vinorelbine (NAVELBINE and others), or pemetrexed (ALIMTA). Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for lung cancer-positive.

As used herein, the term “logistic regression” refers to a regression method in which the assignment of a prediction from the model can have one of several allowed discrete values. For example, the logistic regression models used herein can assign a prediction, for a certain subject, of either lung cancer-positive or lung cancer-negative.

As used herein, the term “biomarker score” refers to a numerical score for a particular subject that is calculated by inputting the particular biomarker levels for said subject to a statistical method.

As used herein, the term “composite score” refers to a summation of the normalized values for the predetermined markers measured in the sample from the subject. In one embodiment, the normalized values are reported as a biomarker score and those biomarker score values are then summed to provide a composite score for each subjected tested. When used in the context of the risk categorization table and correlated to a stratified grouping based on a range of composite scores in the Risk Categorization Table, the “composite score” is used to determine the “risk score” for each subject tested wherein the multiplier indicating increased likelihood of having the cancer for the stratified grouping becomes the “risk score”.

As used herein, the term “risk score” refers to a single numerical value that indicates an asymptomatic human subject's increased risk for harboring a cancer as compared to the known prevalence of cancer in the disease cohort. In certain embodiments, the composite score as calculated for a human subject and correlated to a multiplier indicating increased risk of harboring the cancer, wherein the composite score is correlated based on the range of composite scores for each stratified grouping in the risk categorization table. In this way the composite score is converted to a risk score based on the multiplier indicating increased likelihood of having the cancer for the grouping that is the best match for the composite score.

As used herein, the term “cutoff” or “cutoff point” refers to a mathematical value associated with a specific statistical method that can be used to assign a classification of lung cancer-positive of lung cancer-negative to a subject, based on said subject's biomarker score.

As used herein, when a numerical value above or below a cutoff value “is characteristic of lung cancer,” what is meant is that the subject, analysis of whose sample yielded the value, either has lung cancer or is at risk of harboring lung cancer.

As used herein, a subject who is “at risk of harboring lung cancer” is one who may not yet evidence overt symptoms of lung cancer, but who is producing levels of biomarkers which indicate that the subject has lung cancer, or may develop it in the near term. A subject who has lung cancer or is suspected of harboring lung cancer may be treated for the cancer or suspected cancer.

As used herein, the term “classification” refers to the assignment of a subject as either lung cancer-positive or lung cancer-negative, based on the result of the biomarker score that is obtained for said subject.

As used herein, the term “lung cancer-positive” refers to an indication that a subject is predicted as at risk of harboring lung cancer, based on the results of the outcome of the methods of the disclosure.

As used herein, the term “lung cancer-negative” refers to an indication that a subject is predicted as not at risk of harboring lung cancer, based on the results of the outcome of the methods of the disclosure.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or presence of lung cancer in subjects of a certain population.

As used herein, the term “true positive rate” refers to the probability that a given subject classified as positive by a certain method is truly positive.

As used herein, the term “false positive rate” refers to the probability that a given subject classified as positive by a certain method is truly negative.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, an “antigen” refers to a protein, metabolite, or other molecule to which an antibody or antigen-binding reagent or fragment may bind for detection of a biomarker as described herein. In some embodiments, a biomarker may serve as an antigen. In other embodiments, a portion of a biomarker may serve as an antigen. In some embodiments, an antibody may be used for detection of an antigen as described herein. In other embodiments, a nuceic acid, such as DNA, RNA, DNR/RNA hybrids, antibodies, antibody fragments, or any other compound or molecule capable of binding to an antigen, may be used to detect an antigen, such as a biomarker as described herein. An antigen as described herein may serve as the basis for detection of the levels, concentrations, or amounts of a protein or metabolite marker for use with the methods as described herein.

As used herein, the term “CEA” refers to carcinoembryonic antigen.

As used herein, the term “CA125” refers to cancer antigen 125.

As used herein, the term “CYFRA21-1,” also known as Cyfra 21-1, refers to cytokeratin fragment 19, also known as cytokeratin-19 fragment.

As used herein, the term “SFTPB” refers to Surfactant Protein B.

As used herein, the term “Pro-SFTPB,” refers to Pro-Surfactant Protein B, which is a precursor form of SFTPB.

As used herein, the terms “miR-320,” “miR-210,” and “miR-21” refer to specific microRNAs known in the art. In some embodiments, these miRNAs may be useful for enhancing detection of lung cancer in biological samples of patients suspected as having lung cancer. In some embodiments, miR-320 and miR-210 may be useful for combining with the 4MP in detecting lung cancer in patients. Such combination increases the sensitivity and specificity for detecting lung cancer, which can be described using the area under the curve (AUC, see below). An increase in the AUC for a given marker, panel of markers, or combination of marker panels as described herein, e.g., the 2-miRNA panel or the 3-miRNA panel, combined with the 4MP, indicates that that panel or combination of markers or panels has increased sensitivity and/or specificity when compared to a control sample.

In some embodiments, miRNAs or miRNA panels may be used as markers to detect, or enhance detection of, lung cancer in a patient suspected of having lung cancer. In some embodiments, more than one or multiple miRNA markers may be combined together for use as a diagnostic panel as described herein. In some embodiments, a miRNA or miRNA panel as described herein may be combined with one or metabolite markers, or a metabolite panel, such as a panel including, e.g., diacetylspermine, diacetyspermidine, acetylspermidine, 1-methyladenosine, n-acetyllactosamine, arginine, and dimethyl-arginine. In some embodiments, a miRNA marker or miRNA panel may be combined with the 4MP as described herein for enhanced detection of lung cancer.

Combination of a miRNA panel, such as the 2-miRNA panel or the 3-miRNA panel described herein, with the 4MP, results in an improvement in sensitivity of detection of 5%, or 6%, or 7%, or 8%, or 9%, or 10%, 11%, or 12%, or 13%, or 14%, or 15%, or 16%, or 17%, or 18%, or 19%, or 20%, or 21%, or 22%, or 23%, or 24%, or 25%, 30%, or 40%, or 45%, or 50%, or the like, at 95% specificity. In some embodiments, such a combination may result in an increase in sensitivity of 11%. In some embodiments, combination of a miRNA panel as described herein with the 4MP results in an increase or improvement in the AUC when compared to the 4MP alone. For example, combining the 2-miRNA panel or the 3-miRNA panel described herein with the 4MP may result in an increase or improvement in the AUC of, e.g., 1%, or 2%, or 3%, or 4%, or 5%, or 6%, or 7%, or 8%, or 9%, or 10%, or 15%, or 20%, or 25%, or 30%, or 35%, or 40%, or 45%, or 50%, or the like. In some embodiments, such a combination may result in an increase or improvement in the AUC of 5% or more, or 7% or more, or 8% or more, or the like.

Combination of a miRNA panel, such as the 2-miRNA panel or the 3-mRNA panel described herein, with the 4MP, results in an AUC of, e.g., at least 0.70, or 0.71, or 0.72, or 0.73, or 0.74, or 0.75, or 0.76, or 0.77, or 0.78, or 0.79, or 0.80, or 0.81, or 0.82, or 0.83, or 0.84, or 0.85, or 0.86, or 0.87, or 0.88, or 0.89, or 0.90, or 0.91, or 0.92, or 0.93, or 0.94, or 0.95, or 0.96, or 0.97, or 0.98, or 0.99, or the like. In some embodiments, such a combination may result in an AUC of at least 0.81 compared to an AUC for the 4MP alone.

As used herein, a “metabolite” refers to a substance made or used when the body breaks down food, drugs, or chemicals, or its own tissue (e.g., fat or muscle tissue). Metabolites also help get rid of toxic substances in the body. A metabolite as used herein may be, e.g., diacetylspermine (DAS), diacetyspermidine, acetylspermidine, 1-methyladenosine, n-acetyllactosamine, arginine, and dimethyl-arginine. In some embodiments, metabolites may be used as markers to detect, or enhance detection of, lung cancer in a patient suspected of having lung cancer. In some embodiments, more than one or multiple metabolite markers may be combined together for use as a diagnostic panel as described herein. In some embodiments, a metabolite or metabolite panel as described herein may be combined with one or miRNAs, or a miRNA panel, such as a panel including, e.g., miR-320, miR-210, and/or miR-21. In some embodiments, a metabolite or metabolite panel as described herein may be combined with a miRNA panel including, e.g., miR-320 and miR-210. In some embodiments, a metabolite or metabolite panel may be combined with the 4MP as described herein for enhanced detection of lung cancer.

Combination of a metabolite panel, such as the 7-metabolite panel described herein, with the 4MP, results in an AUC of, e.g., at least 0.70, or 0.71, or 0.72, or 0.73, or 0.74, or 0.75, or 0.76, or 0.77, or 0.78, or 0.79, or 0.80, or 0.81, or 0.82, or 0.83, or 0.84, or 0.85, or 0.86, or 0.87, or 0.88, or 0.89, or 0.90, or 0.91, or 0.92, or 0.93, or 0.94, or 0.95, or 0.96, or 0.97, or 0.98, or 0.99, or the like. In some embodiments, such a combination may result in an AUC of at least 0.88, or at least 0.83, or at least 0.81, or at least 0.80. In some embodiments, combination of a metabolite panel as described herein with the 4MP results in an increase or improvement in the AUC when compared to the 4MP alone. For example, combining the 7-metabolite panel described herein with the 4MP may result in an increase or improvement in the AUC of, e.g., 1%, or 2%, or 3%, or 4%, or 5%, or 6%, or 7%, or 8%, or 9%, or 10%, or 15%, or 20%, or 25%, or 30%, or 35%, or 40%, or 45%, or 50%, or the like. In some embodiments, such a combination may result in an increase or improvement in the AUC of 5% or more, or 7% or more, or 8% or more, or the like.

As used herein, the term “ctDNA” refers to cell-free or circulating tumor DNA. ctDNA is tumor DNA found circulating freely in the blood of a cancer patient. Without being limited by theory, ctDNA is thought to originate from dying tumor cells and can be present in a wide range of cancers but at varying levels and mutant allele fractions. Generally, ctDNA carry unique somatic mutations formed in the originating tumor cell and not found in the host's healthy cells. As such, the ctDNA somatic mutations can act as cancer-specific biomarkers.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, acetylspermidine, diacetylspermine, lysophosphatidylcholine (18:0), lysophosphatidylcholine (20:3), and an indole-derivative.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for lung cancer-positive and lung cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1

### Sources of Study Populations

The Cooper lung nodule and cancer proteomics and genomics research registry was approved by the University of Pittsburgh IRB. The protocol enrolled patients with a confirmed benign lung nodule or diagnosed lung cancer from the Medical Oncology, Thoracic Surgery, and Pulmonary Medicine Clinics. The protocol authorized blood collections for research at periodic intervals, including before and at the time of diagnosis, after surgery, and at the time of lung cancer recurrence. Since 2004, this protocol enrolled 666 patients, with 521 of them eventually diagnosed with lung cancer and the remaining 145 with pulmonary benign nodules.

The Pittsburgh Lung Screening Study (PLuSS) Cohort was approved by the University of Pittsburgh IRB. PLuSS is a community-based research cohort that during 2002-2016 recruited 3,642 smokers (current or former), from 2002-2006.6 Each PLuSS participant completed a questionnaire, underwent spirometry for pulmonary function testing (PFT), received a chest low-dose CT exam, and provided a blood sample. All of the 3,642 participants received a baseline low-dose CT scan, and 3,423 participants received a follow-up low-dose CT scan one-year later. Beginning in 2006, we re-enrolled original participants of PLuSS with the highest lung cancer risk (referenced as PLuSS X). Overall 970 individuals were enrolled into the PLuSS X, who received biennial low-dose CT scans, spirometry and blood draws during 2006-2015. The PluSS X and Cooper registry used the same protocol for blood sample collection, processing and storage.

Blood samples were also obtained through a similar prospective protocol at the University of Texas at Southwestern. 196 total patients presenting for evaluation of pulmonary nodules were enrolled into this protocol. Samples met PRoBE requirements for optimal biomarker collection. Seven patients failed screening, for a total initial enrollment of 186. Of these, 33 were known lung cancer patients, 62 were eventually diagnosed with lung cancer, 40 were negative in a lung cancer workup, and 5 were diagnosed with another cancer, with the rest with no eventual diagnosis. From this, a cohort of 32 patients with lung cancer and 32 controls, matched for age and gender, was assembled.

### Design of UPMC and UTSW Case-Control Study

The UPMC cohort included 100 patients with early stage lung cancer. The median maximum nodule size on diagnostic CT scan was 20 mm (ranging from 7.5 to 38 mm) at initial diagnosis. For each case, we selected one control subject with a similar nodule size (maximum nodule size: 6.0 to 39.0 mm). The selected control was matched to index case by smoking status at the time of blood draw and gender. If a perfect match could not be identified, we dropped the gender as a matching criterion. Due to a small pool of nodule controls available from the Cooper registry, we also selected nodule controls from the PLuSS X participants. Despite attempts, perfect matching in nodule size between case and control cohorts was not achieved across the cohort. For the present study, we selected a plasma sample that was collected within 6 months prior to CT scan that revealed a pulmonary nodule of 6-39 mm. All 200 samples were pulled from the biorepository and sent to a test lab. The case-control status of the biospecimens were blinded to the team for biomarker test. A similar approach was performed with the UTSW cohort, with selection of 32 cases and control matched on age and gender. Due to the size of the cohort, there were significant differences in smoking history and nodule size between lung cancer cases and controls.

Biomarker validation adhered to guidelines outlined by the Institute of Medicine (IOM) and the REMARK criteria. Briefly, samples were drawn under a standard operating procedure for venipuncture and aliquoted in a clinical research laboratory adhering to Clinical Laboratory Improvement Amendment (CLIA) guidelines. The 4MP was already validated in a lung cancer screening population and here was tested, with the same coefficients, in two blinded cohorts of a new intended use population of patients with indeterminate pulmonary nodules. Sensitivities and specificities are reported on this population, building on previous analytical validation on our previous study.

### Luminex Bead Based Assay

Human pro-SFTPB, CEA, CYFRA21-1 and CA125 protein markers were quantified using Luminex bead-based immunoassay and the measured fluorescence intensities was measured with a MAGPIX instrument (Luminex Corporation, Austin TX). Pro-SFTPB Luminex assay was developed in-house as a sandwich ELISA using Mouse monoclonal antibodies against the N-terminus of pro-SFTPB. CEA and CA125 were assayed using a multiplex assay from EMD Millipore Corp. CYFRA21-1 was assayed using a single-plex kit from R&D Systems (Minneapolis, MN, USA). Plasma samples were thawed at 4° C. and centrifuged at 1200 g for 10 mins at 4° C. before plating and testing. Samples were diluted 40× for pro-SFTPB, 6× for CEA/CA125 and 2× for CYFRA21-1. Samples were plated and analyzed in a blinded fashion. Each assay plate contained 7 calibration standards and a blank sample in duplicates. Quality controls include spike-in QCs and low/high plasma controls. The inter-plate and intra-plate coefficients of variation were 3% and 3.6% for pro-SFTPB, 3.19% and 10.4% for CEA, 1.33% and 4.4% for CA125 and 5.01% and 13.9% for CYFRA21-1 respectively.

### Statistical Analyses

The ROC curve estimates are empirically based. 95% confidence intervals and standard errors of the AUC estimates as well those referring to the sensitivity (specificity) at a given specificity (sensitivity) are derived using the bootstrap scheme presented in the Appendix. To derive the ROC curves and corresponding AUC estimates for various fixed values of the covariates of interest, such the packyear and the nodule size, we considered a Cox based modeling technique. The details of this method, named HCNS, can be found in Bantis et al. Lifetime Data Anal. 2012; 18(3):364-396. This estimates the baseline cumulative hazard of a marker and then projects it through a Cox model for the desired covariate level. This is done separately for the control and the case group. Using these cumulative hazard estimates we derive the corresponding estimates of the cumulative distributions for both groups. These, in turn, allow us to derive the ROC for the covariate profile Z, by ROC_{Z}(t)=1−F_{cases|Z=z}(F⁻¹_{controls|Z=z}(1−t)).

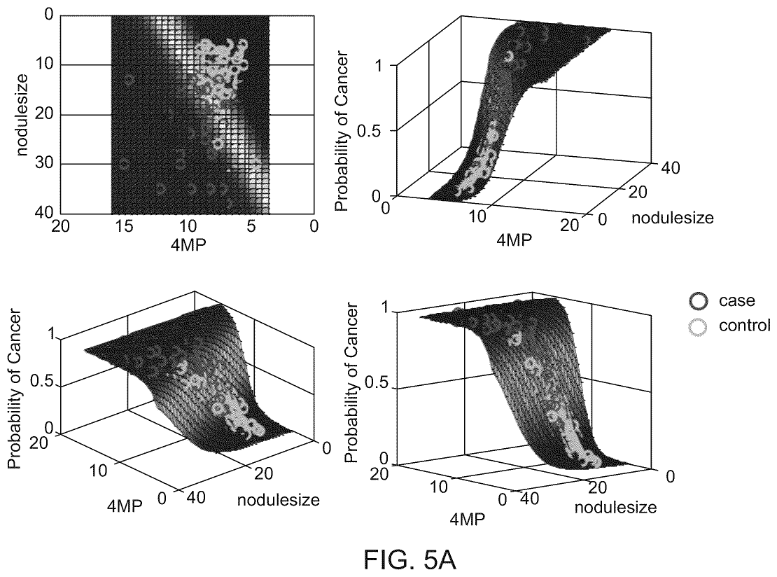

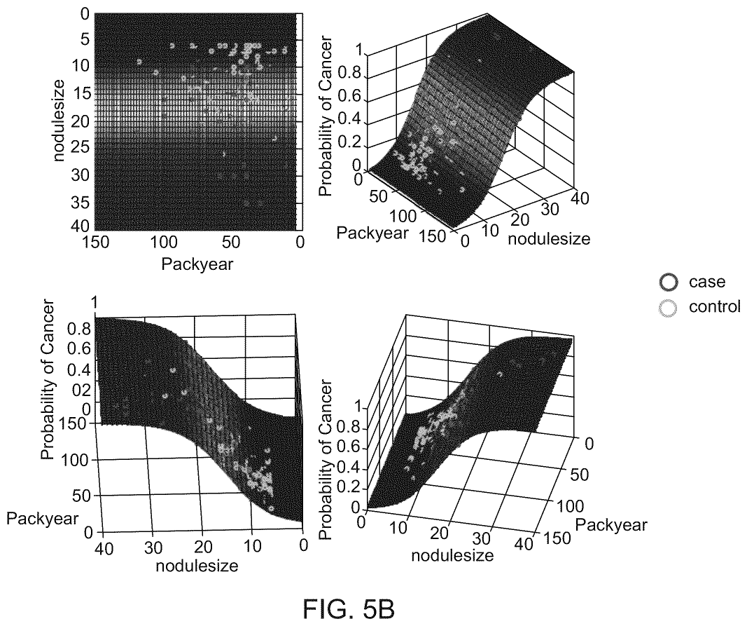

We illustrate both the empirical estimates as well as the corresponding spline-based estimates given by the HCNS method. All corresponding p-values and confidence intervals are derived with the use of the bootstrap. Risks were calculated based on a logistic regression model, using simultaneously both covariates of interest, for instance the pack-year and the nodule size. Such a model induces a risk surface illustrated in FIG. 5A and FIG. 5B, on which the raw data is overlaid and provide the plot from different angles for better visualization. Such a surface allows us to graphically illustrate how steep the risk change is, as either the pack-year or the nodule size value are increased. Numerical results regarding these risks are presented in the results section. The logit link function was used throughout and standard GLM theory is used to fit these logistic regression models.

### Results

### Performance of the 4MP in the Pittsburgh Nodule Cohort

The study design consisted of 200 subjects with pulmonary nodules that were referred to the pulmonary clinic at the University of Pittsburgh Medical Center. The cohort consisted of 100 subjects who were subsequently diagnosed with lung cancer and 100 control patients with benign nodules that were matched for gender, age, and smoking history as shown below.

| | All | | With Cancer | | Without cancer | | p-value |
|---|---|---|---|---|---|---|---|
| Age | 67.7 ± 8.7 | | 68.5 | (Std = 9.2) | 67.0 | (Std = 8.18) | 0.2191 |
| Gender | | | | | | | |
| Female | 88 | | 49 | | 39 | | 0.1543 |
| Male | 112 | | 51 | | 61 | | |
| Smoking status | | | | | | | |
| Current | 74 | | 37 | | 37 | | 1.000 |
| Former | 126 | | 63 | | 63 | | 1.000 |
| Never | 0 | | | | | | |
| Pack years | 48.4 | (Std = 23.1) | 47.8 | (Std = 24.9) | 49.1 | (Std = 21.2) | 0.6926 |
| Nodule size | 16.55 | (Std = 8.47) | 21.5 | (Std = 7.81) | 11.6 | (Std = 5.81) | <0.001 |

(mm)

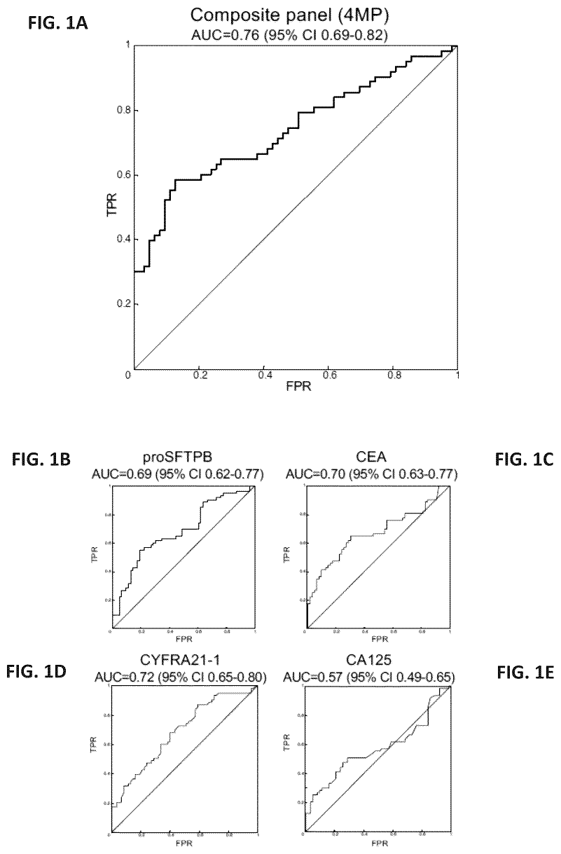

The mean and distributions of age, gender, smoking status, and pack-years of smoking were comparable between cases and controls. The mean (±SD) maximum nodule size was significantly larger for cases (21±7.8 mm) than that of controls (11.6±5.8, p<0.001). Assays of plasmas for the 4MP was performed in a blinded fashion using the same standard operating procedure and fixed coefficients utilized in the pre-diagnostic study. The 4MP showed an area under the curve (AUC) of 0.76, with 95% CI 0.69-0.82 (FIG. 1A). Three of the four independent markers also showed significant discrimination (FIG. 1B-FIG. 1E).

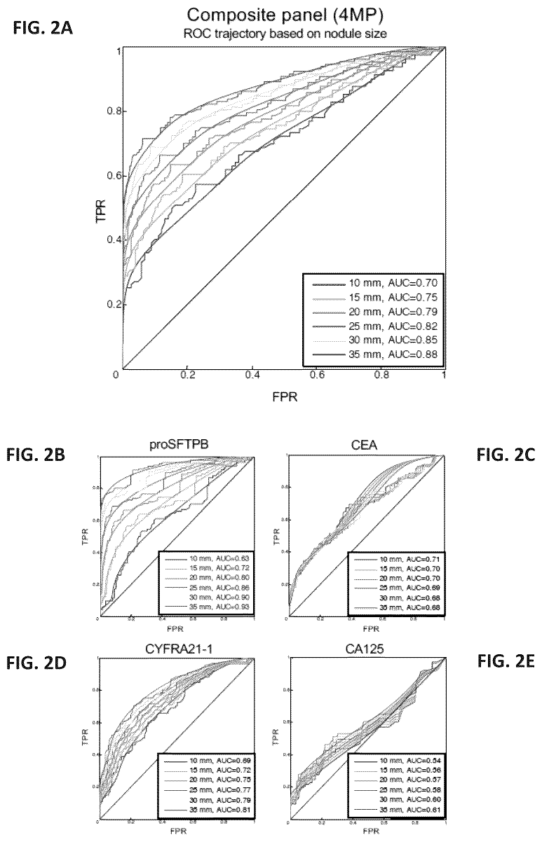

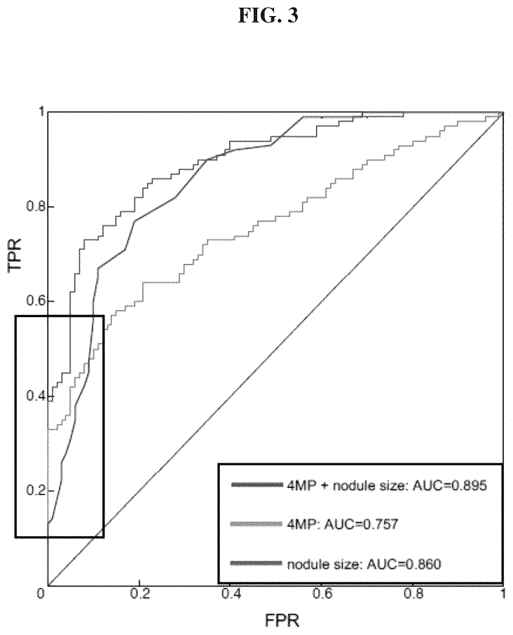

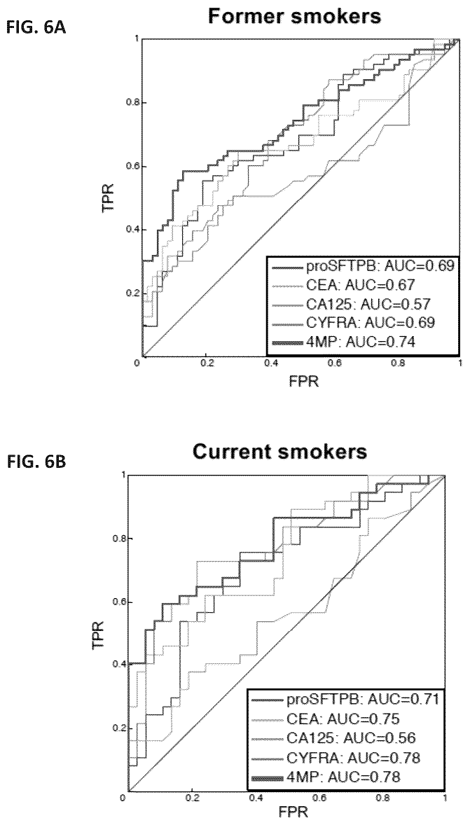

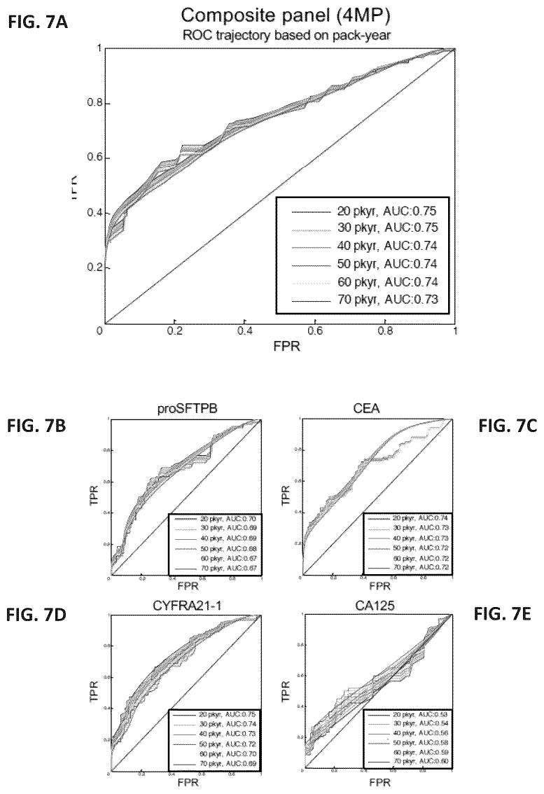

A Cox model accounting for age, gender, smoking history, and nodule size showed significant interaction between the 4MP and nodule size but none of the other variables. There were no significant differences in either the individual markers or the full 4MP between current and former smokers (FIG. 6 and FIG. 7A-FIG. 7E). Pro-SFTPB and CYFRA21-1 exhibited significantly higher performance in larger nodules (FIG. 2A-FIG. 2E). Compared to nodule size alone, the 4MP increased AUC from 0.86 to 0.90 (p-value of the comparison 0.033). Moreover, adding the 4MP to nodule size markedly increased sensitivity at a high specificity. At 99% specificity, the sensitivity of nodule size alone was 14%, which increased to 42% in combination with the 4MP. At 95% specificity, sensitivity increased from 31% to 62%, and at 90% specificity, sensitivity increased from 60% to 73% (FIG. 3).

### Additional Validation in an Independent Cohort

Given the findings in the first validation cohort, we sought to further validate the 4MP in an independent cohort of 60 patients with nodules from the University of Texas Southwestern.

| | All | | With Cancer | | Without cancer | | p-value |
|---|---|---|---|---|---|---|---|
| Age | 59.3 | (Std = 10.7) | 59.9 | (Std = 9.60) | 58.59 | (Std = 11.8) | 0.63 |
| Gender | | | | | | | |
| Female | 30 | | 15 | | 15 | | |
| Male | 34 | | 17 | | 17 | | |
| Smoking status | | | | | | | |
| Current | 39 | | 22 | | 32 | | |
| Former | 20 | | 10 | | 13 | | |
| Never | 5 | | 0 | | 11 | | |
| Pack years | 30.1 | (Std = 33.3) | 44.1 | (Std = 40.2) | 16.6 | (Std = 16.4) | 0.0011 |
| Nodule size | 14.0 | (Std = 15.4) | 19.2 | (Std = 18.8) | 8.40 | (Std = 7.59) | 0.0052 |

(mm)

| | Former Smokers | | | | Current Smokers | | | |
|---|---|---|---|---|---|---|---|---|
| | | p- | | | | p- | | |
| | AUC | value | Se @ | Sp @ | AUC | value | Se @ | Sp @ |
| | (95% | of | 95% | 95% | (95% | of | 95% | 95% |
| | CI) | AUC | Sp | Se | CI) | AUC | Sp | Se |
| proSFTPB | 0.6861 | 0 | 0.2222 | 0.2222 | 0.7071 | 0.0004 | 0.1081 | 0.1351 |
| | (0.5933- | | | | (0.5862- | | | |
| | 0.7788) | | | | 0.828) | | | |
| CEA | 0.6747 | 0.0001 | 0.2698 | 0.0877 | 0.7465 | 0 | 0.3784 | 0.2432 |
| | (0.5818- | | | | (0.6341- | | | |
| | 0.7676) | | | | 0.8589) | | | |
| CA125 | 0.5736 | 0.0748 | 0.254 | 0.0794 | 0.5625 | 0.1703 | 0.1622 | 0.077 |
| | (0.4735- | | | | (0.434- | | | |
| | 0.6736) | | | | 0.6909) | | | |
| CYFRA | 0.69 | 0 | 0.2063 | 0.2722 | 0.775 | 0 | 0.2162 | 0.2122 |
| | (0.5958- | | | | (0.6713- | | | |
| | 0.7841) | | | | 0.8788) | | | |
| Composite | 0.7395 | 0 | 0.3968 | 0.1587 | 0.7816 | 0 | 0.4054 | 0.2162 |
| 4MP | (0.6517- | | | | (0.6758- | | | |
| | 0.8273) | | | | 0.8874) | | | |

The cohort consisted of 30 subjects subsequently diagnosed with lung cancer and 30 subjects with benign nodules matched for age and gender. This cohort had a lower pack-year history and included subjects with smaller nodules. Of the 60 subjects, 27 had nodules <6 mm.

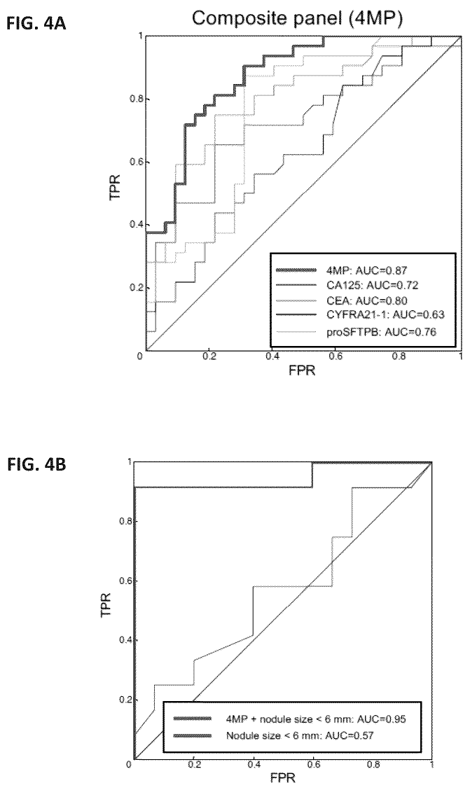

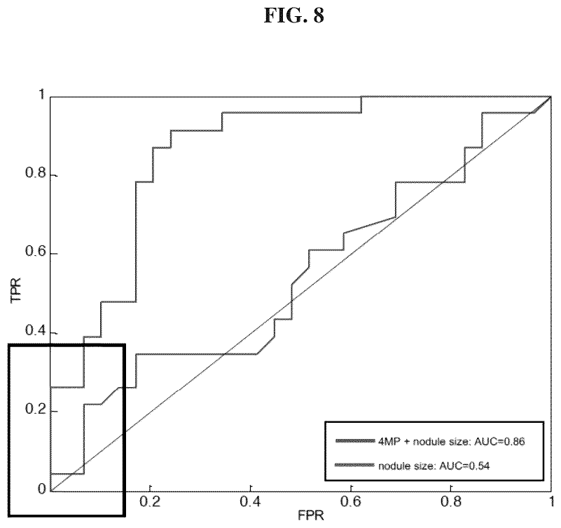

The 4MP performed well in identifying cases of lung cancer in this cohort (FIG. 4A), with an AUC of 0.87 (95% CI 0.79-0.96). Given the higher number of smaller nodules, nodule size poorly predicted cancer risk, with an AUC of the ROC of 0.54 (95% CI 0.37-0.70). Addition of nodule size to the 4MP did not further improve the 4MP performance, with an AUC of 0.86 (95% CI 0.76-0.96). Again, the 4MP significantly improved performance at a 95% specificity, increasing sensitivity from 4% to 26%. (FIG. 8). The performance of the full panel in the subset of 27 subjects with nodule size <6 mm was particularly striking (FIG. 4B). In these patients, with 15 controls and 12 cases, the combination of nodule size and the 4MP showed an AUC of 0.95 with 95% CI 0.85-1.00.

Here, we show that a biomarker panel previously reported to improve a risk prediction in low-dose CT based lung cancer screening also has utility in distinguishing benign from malignant pulmonary nodules. This panel improves the performance of nodule size alone in predicting the risk of cancer in a large cohort of heavy smokers. Notably, the panel improved sensitivity from 14% to 42% at 99% specificity. This points to a potential clinical role in identifying nodules at higher risk. Marker positive nodules could then be followed or biopsied more aggressively, with a high potential for earlier identification and treatment of disease. In a second, smaller cohort, the 4MP performed well again. The fact that we used two different validation cohorts from different institutions with a range of nodule sizes is a strength of our study. Of interest, this second cohort contained 12 cases and 15 controls with nodule size <6 mm. In this small subset, the 4MP performed particularly well, with an AUC of the ROC of 0.95.

### Example 2

The Contributions of 2-miRNA Panel and Metabolites for Detecting Lung Cancer

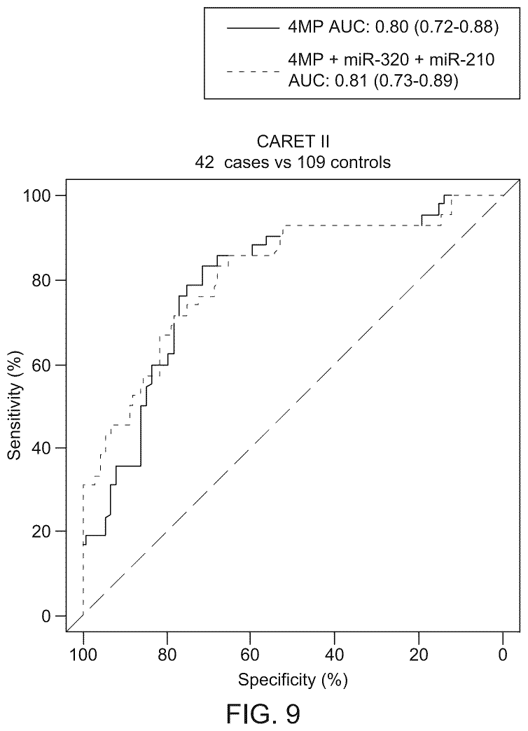

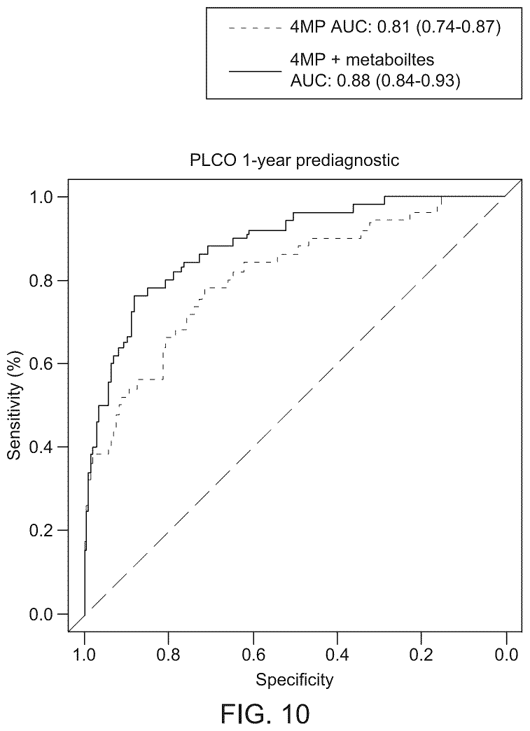

The contributions of microRNAs (miRNAs) and metabolites for detecting lung cancer was evaluated. Quantitative PCR (qPCR)-based validation of several hundred miRNAs in prediagnostic plasma samples from the CARET II trial, resulted in the identification of 3 miRNAs, miR-320, miR-210, and miR-21, that showed significance. Two miRNAs (miR-320 and miR-210) further improved when combined with the 4MP, compared to the 4MP alone, with improvement in sensitivity by 11% at 95% specificity (FIG. 9). Similarly, a panel of 7 cancer-associated metabolites (diacetylspermine, diacetyspermidine, acetylspermidine, 1-methyladenosine, n-acetyllactosamine, arginine, and dimethyl-arginine) have been identified that, in combination with the 4MP, yields an additional improvement in the AUC by 7%, compared to the 4MP alone, for distinguishing cases diagnosed within 1 year of blood draw from non-cases in the PLCO cohort (FIG. 10).

Other analytes may be used to improve the performance of the 4MP. (A) A panel composed of the normalized plasma levels of two miRNAs, miR-210 and miR-320, lends additional discriminatory performance to the 4MP in plasmas drawn within 1-year of diagnosis of lung cancer from the CARET II trial. The miRNA panel was normalized using spike-in controls (cel-miR-39 and cel-miR-54 at 10 fmol) to control for sample-to-sample variation. miRNAs were measured by qRT-PCR with relative quantification to miR-16-5p.

This panel particularly improves sensitivity at a high specificity. (B) A panel of 7 metabolites improves the 4MP in plasmas drawn within 1-year of diagnosis of lung cancer from the PLCO trial.

### Example 3

The Contributions of 3-miRNA Panel and Metabolites for Detecting Lung Cancer

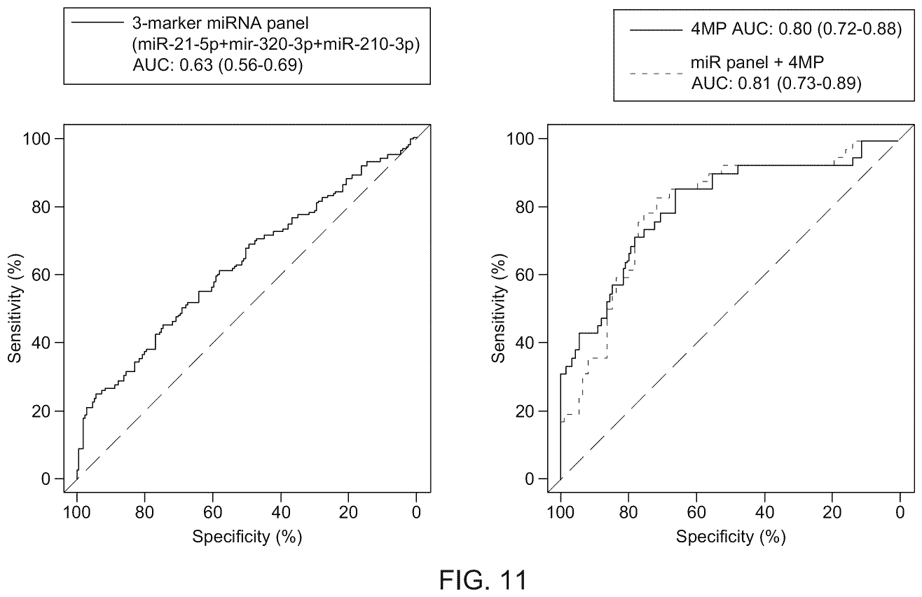

FIG. 11 shows the performance of the 3-miRNA panel, both alone (left graph) and when combined with the 4MP (right graph). The combination of the 3-miRNA panel and the 4MP raises the total area under the curve from 0.8 to 0.81. The miRNA panel was normalized using spike-in controls (cel-miR-39 and cel-miR-54 at 10 fmol) to control for sample-to-sample variation. miRNAs were measured by qRT-PCR with relative quantification to miR-16-5p.

This is a useful trait for an early-detection biomarker to have, as it indicates that positive tests are quite likely to represent true cases. The miRNA panel was normalized using spike-in controls (cel-miR-39 and cel-miR-54 at 10 fmol) to control for sample-to-sample variation. miRNAs were measured by qRT-PCR with relative quantification to miR-16-5p.

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A method of determining the risk of a subject with indeterminate pulmonary nodules for harboring lung cancer, comprising:
measuring the levels of markers CEA, CA125, CYFRA21-1, and Pro-SFTPB, or reporter molecules bound thereto, in a biological sample obtained from the subject;
wherein the combined level of markers CEA, CA125, CYFRA21-1, and Pro-SFTPB classifies the subject as being at risk of harboring lung cancer when the combined level is elevated in the subject relative to a healthy subject,
wherein the biological sample is collected prior to a diagnosis of lung cancer.

2. The method of claim 1, wherein the combined level of markers CEA, CA125, CYFRA21-1, and Pro-SFTPB distinguishes benign pulmonary nodules from malignant pulmonary nodules in the subject.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The method of claim 1, further comprising:
measuring the levels of one or more of markers miR-320, miR-210, and miR-21 in the biological sample; and/or
measuring the levels of one or more of markers diacetylspermine (DAS), diacetyspermidine, acetylspermidine, 1-methyladenosine, n-acetyllactosamine, arginine, and dimethyl-arginine in the biological sample;
wherein the amount of miR-320, miR-210, and/or miR-21, and/or the amount of diacetylspermine (DAS), diacetyspermidine, acetylspermidine, 1-methyladenosine, n-acetyllactosamine, arginine, and/or dimethyl-arginine classifies the patient as being at risk of harboring lung cancer or not at risk of harboring lung cancer or distinguishes the patient as having benign or malignant pulmonary nodules.

37. The method of claim 1, wherein the subject is determined to have lung cancer based on the measured concentrations of the biomarkers.

38. The method of claim 1, further comprising comparing the measured concentrations of each biomarker in the biological sample to the prediction of a statistical model.

39. The method of claim 1, further comprising administering at least one alternate diagnostic test for a subject assigned as having lung cancer.

40. The method of claim 39, wherein the at least one alternate diagnostic test comprises an assay or sequencing of at least one ctDNA.

41. The method of claim 1, wherein the lung cancer is diagnosed at or before the borderline resectable stage, or at the resectable stage.

42. (canceled)

43. (canceled)

44. The method of claim 36, wherein the markers consist of:
CEA, CA125, CYFRA21-1, Pro-SFTPB, and diacetylspermine (DAS); or
miRNA-320 and miRNA-210; or
diacetylspermine (DAS), diacetyspermidine, acetylspermidine, 1-methyladenosine, n-acetyllactosamine, arginine, and dimethyl-arginine; or
miRNA-320, miRNA-210, diacetylspermine (DAS), diacetylspermidine, acetylspermidine, 1-methyladenosine, n-acetyllactosamine, arginine, and dimethyl-arginine.

45. (canceled)

46. (canceled)

47. (canceled)

48. The method of claim 1, wherein the markers comprise a panel selected from the group consisting of:
a. the panel consisting of CEA, CA125, CYFRA21-1, and Pro-SFTPB; or
b. the panel consisting of CEA, CA125, CYFRA21-1, Pro-SFTPB, and diacetylspermine (DAS); or
c. the panel consisting of miRNA-320 and miRNA-210; or
d. the panel consisting of diacetylspermine (DAS), diacetyspermidine, acetylspermidine, 1-methyladenosine, n-acetyllactosamine, arginine, and dimethyl-arginine; or
e. any combination of a-d.

49. (canceled)

50. The method of claim 48, wherein:
the levels of CEA, CA125, CYFRA21-1, and pro-SFTPB are elevated in the subject relative to a healthy subject; and/or
the levels of miR-320, miR-210, and/or miR-21 are reduced in the subject relative to a healthy subject; and/or
the levels of diacetylspermine (DAS), diacetyspermidine, acetylspermidine, 1-methyladenosine, n-acetyllactosamine, arginine, and dimethyl-arginine are elevated in the subject relative to a healthy subject.

51. (canceled)

52. (canceled)

53. The method of claim 50, wherein:
the amount of CEA, CA125, CYFRA21-1, and pro-SFTPB is quantified; and/or
the amount of miR-320, miR-210, and/or miR-21 is quantified; and/or
the amount of diacetylspermine (DAS), diacetyspermidine, acetylspermidine, 1-methyladenosine, n-acetyllactosamine, arginine, and dimethyl-arginine is quantified.

54. (canceled)

55. (canceled)

56. The method of claim 1, wherein:
the concentrations of CEA, CA125, CYFRA21-1, Pro-SFTPB, and diacetylspermine (DAS) are measured; and/or
the concentrations of miR-320, miR-210, and/or miR-21 are measured; and/or
the concentrations of diacetylspermine (DAS), diacetyspermidine, acetylspermidine, 1-methyladenosine, n-acetyllactosamine, arginine, and dimethyl-arginine are measured.

57. (canceled)

58. (canceled)

59. (canceled)

60. (canceled)

61. (canceled)

62. (canceled)

63. (canceled)

64. The method of claim 1, wherein determination of CEA, CA125, CYFRA21-1, and pro-SFTPB levels is made at substantially the same time or in a stepwise manner.

65. (canceled)

66. (canceled)

67. (canceled)

68. (canceled)

69. (canceled)

70. (canceled)

71. The method of claim 1, wherein the sample comprises a biological sample selected from blood, plasma, and serum.

72. (canceled)

73. The method of claim 56, wherein detection of the amount of CEA, CA125, CYFRA21-1, pro-SFTPB, and diacetylspermine (DAS) comprises the use of a solid particle.

74. (canceled)

75. The method of claim 73, wherein the solid particle is a bead.

76. The method of claim 1, wherein at least one of the reporter molecules is linked to an enzyme or provides a detectable signal.

77. (canceled)

78. The method of claim 76, wherein the detectable signal is detectable by a method selected from UV-visible spectroscopy, mass spectrometry, nuclear magnetic resonance (NMR) spectroscopy, proton NMR spectroscopy, nuclear magnetic resonance (NMR) spectrometry, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry.

79. (canceled)

80. (canceled)

81. The method of claim 1, wherein the method further comprises comparing the amount of CEA, CA125, CYFRA21-1, and pro-SFTPB with a cutoff value comprising an AUC (95% CI) of at least 0.83; or at least 0.80; or at least 0.81; or at least 0.88.

82. (canceled)

83. (canceled)

84. (canceled)

85. The method of claim 1, wherein:
the classification of the subject as at risk of having lung cancer has a sensitivity of 73% at 90% specificity, 62% at 95% specificity and/or 42% at 99% specificity; or
the classification of the subject as at risk of having lung cancer has an increase in sensitivity of 11% at 95% specificity compared to control; or
the classification of the subject as at risk of having lung cancer has an increase in the AUC of 7% compared to control.

86. (canceled)

87. (canceled)

88. A method comprising:
a) obtaining a sample from a subject with pulmonary nodules;
b) measuring a panel of markers in the sample, wherein the markers comprise:
CEA, CA125, CYFRA21-1, and diacetylspermine (DAS); and/or
miRNA-320 and miRNA-210; and/or
diacetylspermine, diacetyspermidine, acetylspermidine, 1-methyladenosine, n-acetyllactosamine, arginine, and dimethyl-arginine;
c) determining a biomarker score for each marker;
d) summing the biomarker scores for each marker to obtain a composite score for each subject, quantifying the increased risk for the presence of lung cancer for the subject as a risk score, wherein the composite score is matched to a risk category of a grouping of stratified subject populations, wherein each risk category comprises a multiplier indicating increased likelihood of having the lung cancer correlated to a range of composite scores as compared to use of a single threshold value, wherein the multiplier is determined from positive predictive scores of retrospective samples; and,
e) administering a computerized tomography (CT) scan or other imagine modality to the subject with a quantified increased risk for the presence of lung cancer.

89. The method of claim 88, wherein the grouping of a stratified subject population, the multiplier indicating increased likelihood of having the cancer and the range of composite scores are determined from retrospective clinical samples of a population.

90. The method of claim 88, wherein the risk category further comprises a risk identifier selected from low risk, intermediate-low risk, intermediate risk, intermediate-high risk, and highest risk.

91. (canceled)

92. The method of claim 88, wherein:
the grouping of a stratified subject population comprises at least three risk categories wherein the multiplier indicating increased likelihood of having cancer is about 2 or greater; or
the grouping of a stratified subject population comprises at least two risk categories wherein the multiplier indicating increased likelihood of having cancer is about 5 or greater.

93. (canceled)

94. The method of claim 88, wherein the subject is aged 50 years or older and has a history of smoking tobacco.

95. The method of claim 88, wherein the method further comprises generating a risk categorization table, wherein the panel of markers is measured, a biomarker score for each marker is determined, a composite score is obtained by summing the biomarker scores; determining a threshold value used to divide the composite scores into risk groups and assigning a multiplier to each group indicating the likelihood of an asymptomatic subject having a quantified increased risk for the presence of cancer.

96. (canceled)

97. (canceled)

98. A method of treatment or prevention of progression of lung cancer in a subject with indeterminate pulmonary nodules in whom the levels of CEA, CA125, CYFRA21-1, and pro-SFTPB classifies the subject with indeterminate pulmonary nodules as having or being at risk of harboring lung cancer comprising one or more of:
i. administering a chemotherapeutic drug to the subject with lung cancer;
ii. administering therapeutic radiation to the subject with lung cancer; and
iii. surgery for partial or complete surgical removal of cancerous tissue in the subject with lung cancer.

99. (canceled)

100. A method for detecting and treating lung cancer, comprising:
detecting CEA, CA125, CYFRA21-1, and pro-SFTPB, in a biological sample obtained from a human with indeterminate pulmonary nodules, via an immunoassay;
and/or detecting miRNA-320 and miRNA-210, in a biological sample obtained from a human with indeterminate pulmonary nodules, via an immunoassay; and/or
detecting diacetylspermine (DAS), diacetyspermidine, acetylspermidine, 1-methyladenosine, n-acetyllactosamine, arginine, and dimethyl-arginine, in a biological sample obtained from a human with indeterminate pulmonary nodules, via an immunoassay; and/or
quantifying the amounts of CEA, CA125, CYFRA21-1, and pro-SFTPB in said collected sample; and/or
quantifying the amounts of miRNA-320 and miRNA-210 in said collected sample;
and/or quantifying the amounts of diacetylspermine (DAS), diacetyspermidine, acetylspermidine, 1-methyladenosine, n-acetyllactosamine, arginine, and dimethyl-arginine in said collected sample;
comparing the amounts of CEA, CA125, CYFRA21-1, and pro-SFTPB; and/or the amounts of miRNA-320 and miRNA-210; and/or the amounts of diacetylspermine (DAS), diacetyspermidine, acetylspermidine, 1-methyladenosine, n-acetyllactosamine, arginine, and dimethyl-arginine, with a cutoff value to determine whether said human is at increased risk of having lung cancer or not; wherein if the levels are above the cutoff value said human has lung cancer, and
administering a treatment for lung cancer to said human having lung cancer.

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)
